(19) United States
(12) Patent Application Publication
(10) Pub. No.: US 2020/0282052 A1
(43) Pub. Date: Sep. 10, 2020

(54) A COMBINATION THERAPY WITH APATINIB FOR THE TREATMENT OF CANCER

(71) Applicant: Elevar Therapeutics, Inc., Salt Lake City, UT (US)

(72) Inventors: Arlo N. MCGINN, Salt Lake City, UT (US); Bumjin KIM, Salt Lake City, UT (US); Sung Chul KIM, Salt Lake City, UT (US); Cheol Hee PARK, Salt Lake City, UT (US)

(21) Appl. No.: 16/762,881

(22) PCT Filed: Nov. 9, 2018
(86) PCT No.: PCT/US2018/060179

§ 371 Date: May 8, 2020

Related U.S. Application Data
(60) Provisional application No. 62/584,547, filed on Nov. 10, 2017.

(51) Int. Cl. A61K 39/395; A61K 31/444; A61K 9/00; A61K 45/06; A61K 9/28

(52) U.S. Cl. CPC A61K 39/3955; A61K 31/444; A61K 9/28; A61K 45/06; A61K 9/0053

(57) ABSTRACT

Provided herein are methods for the treatment of diseases, comprising administering a combination of a tyrosine kinase inhibitors and an immunotherapeutic agent. A method of treating cancer comprising administering to a patient in need thereof a therapeutically effective amount of apatinib in combination with a therapeutically effective amount of an immunotherapeutic agent.

### CROSS-REFERENCE

This application claims the benefit of U.S. Provisional Application No. 62/584,547, filed Nov. 10, 2017, which application is incorporated herein by reference.

### BACKGROUND OF THE DISCLOSURE

Tumor angiogenesis plays a critical role in malignant tumor growth and metastasis. When tumors grow beyond 1 mm³, angiogenesis, or generation of vascular arborizations by budding from existing vessels, is necessary to provide enough blood for the survival of tumor cells. The growth speed and tendency of metastasis of tumors are associated with the level of neovascularization factors and the quantity of nascent microvessels. Since the hypothesis of “anti-angiogenesis therapy” was put forward by Folkman in early 1970s, people have made considerable progress in this field, and inhibiting angiogenesis of tumors has been universally accepted as a promising anticancer strategy.

Recently, immunotherapy has led to unprecedented improvements in overall survival in the patients of various cancer types including non-small cell lung cancer, urothelial carcinoma, melanoma, and head and neck carcinoma. In particular, reversing immune-suppressive tumor microenvironment via blocking interactions of immune checkpoint molecules such as programmed death-1 (PD-1), programmed death-ligand 1 (PD-L1), and cytotoxic T lymphocyte-associated antigen 4 (CTLA-4) have been recognized as promising strategies in immunotherapy. This is because solid tumors are capable of escaping the surveillance of host immune system through “hijacking” the locally-expressed immune checkpoint molecules. However, the effect of targeting the immune checkpoint molecules alone may be insufficient, especially for advanced or intractable malignant tumors. From clinical trials, it is known that a subset of patients (up to approximately 80% in most cancers) do not respond to monotherapy. Therefore, it is important to investigate combination treatments for augmenting the potency of immune checkpoint inhibitors.

Beyond the tumor-growth-limiting effect of anti-angiogenesis therapy, it has at least two additional mechanisms that allow for synergistic effect when combined with tumor immunotherapy: 1) the normalization of tumor vasculature, and 2) the amelioration of immune-suppressive tumor microenvironment.

Tumor vasculature is highly chaotic and unnatural due to the supra-physiological levels of proangiogenic factors and the lack of balancing with their anti-angiogenic counterparts. Vessels formed by tumors are immature, and leaky, with endothelial cells that express lower levels of chemokines, and adhesion factors, which are responsible for recruiting activated immune cells. Consequently, these features limit the ability of immune cells to effectively infiltrate into the tumor and achieve extravasation.

Not only does anti-angiogenesis therapy help immune cells infiltrate into the tumor, but it also helps create a more favorable tumor microenvironment for immunotherapy. High levels of proangiogenic factors are known to create a local environment that is extremely hostile towards immune cells, allowing tumors to evade host immune surveillance. Indeed, angiogenic factors such as vascular endothelial growth factor (VEGF), which is overexpressed in tumor tissues are known to induce the secretion of immune-suppressive cytokines and to recruit regulatory T-cells (T_{reg}), tumor-associated macrophages, sTie2-expressing monocytes, and myeloid-derived suppressor cells (MDSCs), all of which are responsible for immune suppression. Anti-angiogenesis therapy, however, reverses these effects, leading to increased anti-tumor immunity.

There is, therefore, a need for new combinational treatments with immunotherapeutic compounds.

Rivoceranib (also known as YN968D1, developed in China as apatinib and marketed as Aitan®) is an orally administered small molecule tyrosine kinase inhibitor with selectivity towards the VEGFR-2/kinase insert domain receptor. Rivoceranib has received approval in China, for treatment of advanced gastric cancer, and has received orphan medicinal product designation for the treatment of gastric cancer from Europe, the FDA, and the MFDS in South Korea. According to a recent review (see L. J. Scott, “Apatinib: A Review in Advanced Gastric Cancer and Other Advanced Cancers,” Drugs, 2018, 78(7), 747-758), “further clinical experience and long-term pharmacovigilance data are required to more definitively establish the efficacy and safety profile of apatinib, including its use in combination with other chemotherapy agents . . . .”

Nivolumab is an immunotherapeutic agent, approved for the treatment of non-small cell lung cancer, advanced small cell lung cancer, metastatic melanoma, kidney cancer (renal cell carcinoma), advanced renal cell carcinoma, squamous cell carcinoma, liver cancer, (hepatocellular carcinoma), bladder cancer (urothelial carcinoma), colon cancer, and Hodgkin lymphoma. It is administered by intravenous injection.

Pembrolizumab is an immunotherapeutic agent, approved for the treatment of melanoma, non-small cell lung cancer. In some embodiments, permbrolizumab is used to treat metastatic melanoma, metastatic non-small cell lung cancer (NSCLC) in certain situations, as a second-line treatment for head and neck squamous cell carcinoma (HNSCC), after platinum-based chemotherapy, and for the treatment of adult and pediatric patients with refractory classic Hodgkin's lymphoma It is administered by intravenous injection.

There exists a need for an effective treatment of cancer and neoplastic diseases.

There exists a need for effective treatment of cancer with fewer side effects.

There exists a need for effective treatment of cancer with less severe side effects.

There exists a need for effective treatment of cancer allowing for administration of lower doses.

There exists a need for effective treatment of cancer with longer survival rates.

There exists a need for effective treatment of cancer with more convenient administration methods.

There exists a need for combination therapy modalities to treat cancer and other diseases.

### SUMMARY OF THE DISCLOSURE

In one aspect, the disclosure provides a combination therapy comprising apatinib, or a pharmaceutically acceptable salt thereof, and an immunotherapeutic agent.

The disclosure also provides a method of treating cancer comprising administering to a patient in need thereof a therapeutically effective amount of apatinib in combination with a therapeutically effective amount of an immunotherapeutic agent.

In one aspect, the pharmaceutically acceptable salt is apatinib mesylate.

In one aspect, the immunotherapeutic agent is selected from the group consisting of an antibody, a peptide, pembrolizumab, nivolumab, pidilizumab, BMS-936559, MPDL3280A, MEDI4736, MSB0010718C, atezolizumab, avelumab, durvalumab, ipilimumab, tumor vaccines (e.g., sipuleucel-T), CAR T-cell therapies (e.g., tisagenlecleucel, axicabtagene ciloleucel), and naked monoclonal antibodies (e.g., alemtuzumab).

Cancers treatable by the combination therapies of the disclosure include, but are not limited to lung cancer, small-cell lung cancer, non-small cell lung cancer, carcinoma, lymphoma, blastoma, sarcoma, leukemia, breast cancer, prostate cancer, colon cancer, squamous cell cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

In one aspect, the immunotherapeutic agent is an antibody.

In one aspect, the antibody can be a monoclonal or polyclonal antibody.

In one aspect, the antibody can be a human or humanized antibody.

In one aspect, the antibody can be an anti-programmed death 1 (PD-1) antibody.

In one aspect, the antibody can be an anti-muPD-1 antibody.

In one aspect, the antibody can be an anti-PD-L1 antibody.

In one aspect, the antibody can be an anti-CTLA-4 antibody.

In some embodiments, disclosed herein is a method for treating cancer, comprising administering: a) rivoceranib, or a pharmaceutically acceptable salt thereof; and b) pembrolizumab. In some embodiments, disclosed herein is a method for treating cancer, comprising administering: a) rivoceranib, or a pharmaceutically acceptable salt thereof; and b) nivolumab. In some embodiments, a pharmaceutically acceptable salt of rivoceranib is administered. In some embodiments, the pharmaceutically acceptable salt of rivoceranib is a mesylate salt. The In some embodiments, the rivoceranib is administered in an amount of from 100 mg to 1000 mg. In some embodiments, the rivoceranib is administered in an amount of from 150 mg to 800 mg. In some embodiments, the rivoceranib is administered in an amount of from 200 mg to 700 mg. In some embodiments, the rivoceranib is administered in an amount of less than 700 mg. In some embodiments, the rivoceranib is administered in an amount of about 200 mg. In some embodiments, the rivoceranib is administered in an amount of about 300 mg. In some embodiments, the rivoceranib is administered in an amount of about 400 mg. In some embodiments, the rivoceranib is administered in an amount of about 500 mg. In some embodiments, the rivoceranib is administered in an amount of about 600 mg. In some embodiments, the rivoceranib is administered in an amount of about 685 mg. In some embodiments, the total daily dose of the rivoceranib is less than 700 mg. In some embodiments, the total daily dose of the rivoceranib is less than 685 mg. In some embodiments, the rivoceranib is administered orally. In some embodiments, the rivoceranib is administered as a dried powder, a liquid, a capsule, a pellet or a tablet. In some embodiments, the rivoceranib is administered as a tablet. In some embodiments, the tablet is a film coated tablet. In some embodiments, the tablet comprises the rivoceranib in an amount of about 100 mg. In some embodiments, the tablet comprises the rivoceranib in an amount of about 200 mg. In some embodiments, the tablet further comprises one or more of pregelatinized starch, microcrystalline cellulose, sodium starch glycolate, povidone (K-30), colloidal silicon dioxide, magnesium stearate and Opadry white. In some embodiments, the rivoceranib is administered once daily. In some embodiments, the rivoceranib is administered twice daily. In some embodiments, disclosed herein is a method for treating cancer, comprising administering: a) rivoceranib, or a pharmaceutically acceptable salt thereof; and b) pembrolizumab. In some embodiments, the pembrolizumab is administered in a dosage amount of about 200 mg. In some embodiments, the pembrolizumab is administered in a dosage amount of 150 mg to 250 mg. In some embodiments, the pembrolizumab is administered orally or parenterally. In some embodiments, the pembrolizumab is administered parenterally. In some embodiments, the parenteral administration is selected from intravenous, intradermal, intramuscular or subcutaneous administration. In some embodiments, the pembrolizumab is administered intravenously. In some embodiments, the pembrolizumab is administered after the administration of the rivoceranib. In some embodiments, the pembrolizumab is administered about an hour after the administration of the rivoceranib. In some embodiments, the pembrolizumab is administered over a period of less than one hour. In some embodiments, the pembrolizumab is administered over a period of about an hour. In some embodiments, the pembrolizumab is administered over a period of 30-60 minutes. In some embodiments, the pembrolizumab is administered no more than once a week. In some embodiments, the pembrolizumab is administered at least once a week. In some embodiments, the pembrolizumab is administered once a week. In some embodiments, the pembrolizumab is administered every three weeks. In some embodiments, the pembrolizumab is administered once a month. In some embodiments, the pembrolizumab is administered twice a month. In some embodiments, the pembrolizumab is administered three times a month. In some embodiments, disclosed herein is a method for treating cancer, comprising administering: a) rivoceranib, or a pharmaceutically acceptable salt thereof; and b) nivolumab. In some embodiments, the nivolumab is administered in a dosage amount of about 240 mg. In some embodiments, the nivolumab is administered in a dosage amount of 200 mg to 300 mg. In some embodiments, the nivolumab is administered orally or parenterally. In some embodiments, the nivolumab is administered parenterally. In some embodiments, the parenteral administration is selected from intravenous, intradermal, intramuscular or subcutaneous administration. In some embodiments, the nivolumab is administered intravenously. In some embodiments, the nivolumab is administered after the administration of the rivoceranib. In some embodiments, the nivolumab is administered about an hour after the administration of the rivoceranib. In some embodiments, the nivolumab is administered over a period of less than one hour. In some embodiments, the nivolumab is administered over a period of about an hour. In some embodiments, the nivolumab is administered over a period of 30-60 minutes. In some embodiments, the nivolumab is administered no more than once a week. In some embodiments, the nivolumab is administered at least once a week. In some embodiments, the nivolumab is administered every two weeks. In some embodiments, the nivolumab is administered once a week. In some embodiments, the nivolumab is administered once a month. In some embodiments, the nivolumab is administered twice a month. In some embodiments, the nivolumab is administered three times a month. In some embodiments, the cancer is selected from lung cancer, small-cell lung cancer, non-small cell lung cancer, carcinoma, lymphoma, blastoma, sarcoma, leukemia, breast cancer, prostate cancer, colon cancer, squamous cell cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer. In some embodiments, the cancer is melanoma or non-small cell lung cancer. In some embodiments, the cancer is non-small cell lung cancer, advanced small cell lung cancer, metastatic melanoma, kidney cancer (renal cell carcinoma), advanced renal cell carcinoma, squamous cell carcinoma, liver cancer, (hepatocellular carcinoma), bladder cancer (urothelial carcinoma), colon cancer, or Hodgkin lymphoma. In some embodiments, the method further comprises administering radiation therapy. In some embodiments, the cancer comprises a lesion. In some embodiments, the lesion is measured before the treatment and either during the treatment or after the treatment or both. In some embodiments, the lesion is measured by radiological assessments using computerized tomography scan or magnetic resonance imaging. In some embodiments, the lesion has reduced in size after the treatment. In some embodiments, the lesion has reduced in size by at least 10%. In some embodiments, the lesion has reduced in size by at least 20%. In some embodiments, the lesion has reduced in size by at least 50%. In some embodiments, the lesion has reduced in size by at least 75%.

In some embodiments, disclosed herein is a method for treating cancer, comprising administering a combination of: a) a tyrosine kinase inhibitor, or a pharmaceutically acceptable salt thereof; and b) an immunotherapeutic agent, or a pharmaceutically acceptable salt thereof. In some embodiments, the tyrosine kinase inhibitor is a vascular endothelial growth factor receptor (VEGF) inhibitor. In some embodiments, the tyrosine kinase inhibitor is a selective vascular endothelial growth factor receptor-2 (VEGF2) inhibitor. In some embodiments, the tyrosine kinase inhibitor is afatinib, alectinib, apatinib, axitinib, bosutinib, brigatinib, canertinib, crizotinib, ceritinib, dasatinib, danusertib, dabrafenib, erlotinib, gefitinib, ibrutinib, imatinib, lapatinib, lenvatinib, neratinib, nilotinib, nintedanib, osimertinib, palbociclib, pazopanib, pegaptanib, ponatinib, rebastinib, regorafenib, ribociclib, rivoceranib, ruxolitinib, semaxinib, sorafenib, sunitinib, tivozanib, trametinib, tofacitinib, vandetanib, vatalanib, vemurafenib or vismodegib. In some embodiments, the tyrosine kinase inhibitor is rivoceranib. In some embodiments, the tyrosine kinase inhibitor is rivoceranib mesylate. In some embodiments, the tyrosine kinase inhibitor is administered in an amount of from 150 mg to 800 mg. In some embodiments, the tyrosine kinase inhibitor is administered in an amount of from 200 mg to 700 mg. In some embodiments, the tyrosine kinase inhibitor is administered in an amount of less than 700 mg. In some embodiments, the tyrosine kinase inhibitor is administered in an amount of about 200 mg. In some embodiments, the tyrosine kinase inhibitor is administered in an amount of about 300 mg. In some embodiments, the tyrosine kinase inhibitor is administered in an amount of about 400 mg. In some embodiments, the tyrosine kinase inhibitor is administered in an amount of about 500 mg. In some embodiments, the tyrosine kinase inhibitor is administered in an amount of about 600 mg. In some embodiments, the total daily dose of the tyrosine kinase inhibitor is less than 700 mg. In some embodiments, the total daily dose of the tyrosine kinase inhibitor is less than 685 mg. In some embodiments, the tyrosine kinase inhibitor is administered orally. In some embodiments, the tyrosine kinase inhibitor is administered as a tablet. In some embodiments, the tyrosine kinase inhibitor is administered once daily. In some embodiments, the tyrosine kinase inhibitor is administered twice daily. In some embodiments, the immunotherapeutic agent is a PD-1 inhibitor. In some embodiments, the PD-1 inhibitor is selected from nivolumab (Opdivo®), pembrolizumab (Keytruda®), MEDI0680 (AMP-514), AMP-224, AMP-514 (Amplimmune), BGB-A317, PDR001, REGN2810, JS001, AGEN2034, and variants and biosimilars thereof. In some embodiments, the PD-1 inhibitor is selected from nivolumab (Opdivo®), pembrolizumab (Keytruda®), and variants and biosimilars thereof. In some embodiments, the immunotherapeutic agent is administered in a dosage amount of 200 mg to 300 mg. In some embodiments, the immunotherapeutic agent is administered orally or parenterally. In some embodiments, the immunotherapeutic agent is administered parenterally. In some embodiments, the parenteral administration is selected from intravenous, intradermal, intramuscular or subcutaneous administration. In some embodiments, the immunotherapeutic agent is administered intravenously. In some embodiments, the immunotherapeutic agent is administered after the administration of the tyrosine kinase inhibitor. In some embodiments, the immunotherapeutic agent is administered about an hour after the administration of the tyrosine kinase inhibitor. In some embodiments, the immunotherapeutic agent is administered over a period of less than one hour. In some embodiments, the immunotherapeutic agent is administered over a period of about an hour. In some embodiments, the immunotherapeutic agent is administered over a period of 30-60 minutes. In some embodiments, the immunotherapeutic agent is administered no more than once a week. In some embodiments, the immunotherapeutic agent is administered at least once a week. In some embodiments, the immunotherapeutic agent is administered every two weeks. In some embodiments, the immunotherapeutic agent is administered once a week. In some embodiments, the immunotherapeutic agent is administered once a month. In some embodiments, the immunotherapeutic agent is administered twice a month. In some embodiments, the immunotherapeutic agent is administered three times a month.

In some embodiments, disclosed herein is a method for treating cancer, comprising administering a combination of: a) rivoceranib, or a pharmaceutically acceptable salt thereof; and b) no more than 80 mg/m2 pembrolizumab. In some embodiments, disclosed herein is a method for treating cancer, comprising administering a combination of: a) no more than 685 mg rivoceranib, or a pharmaceutically acceptable salt thereof; and b) pembrolizumab. In some embodiments, disclosed herein is a method for treating cancer, comprising administering a combination of: a) no more than 685 mg rivoceranib, or a pharmaceutically acceptable salt thereof; and b) no more than 80 mg/m2 pembrolizumab. In some embodiments, disclosed herein is a method for treating cancer, comprising administering a combination of: a) rivoceranib, or a pharmaceutically acceptable salt thereof; and b) pembrolizumab; wherein the rivoceranib and the pembrolizumab act synergistically. In some embodiments, disclosed herein is a method for treating cancer, comprising administering a combination of: a) rivoceranib, or a pharmaceutically acceptable salt thereof; and b) no more than 80 mg/m2 nivolumab. In some embodiments, disclosed herein is a method for treating cancer, comprising administering a combination of: a) no more than 685 mg rivoceranib, or a pharmaceutically acceptable salt thereof; and b) nivolumab. In some embodiments, disclosed herein is a method for treating cancer, comprising administering a combination of: a) no more than 685 mg rivoceranib, or a pharmaceutically acceptable salt thereof; and b) no more than 80 mg/m2 nivolumab. In some embodiments, disclosed herein is a method for treating cancer, comprising administering a combination of: a) rivoceranib, or a pharmaceutically acceptable salt thereof; and b) nivolumab; wherein the rivoceranib and the nivolumab act synergistically.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a graph of tumor growth curve demonstrating synergistic anti-tumor efficacy of a combination of apatinib mesylate and anti-muPD-1 antibody on mouse LL/2 lung carcinoma transplanted in syngeneic mice.

FIG. 2 is graph of body weight curve demonstrating negligible toxicity of a combination of apatinib mesylate and anti muPD-1 antibody on mouse LL/2 lung carcinoma transplanted in syngeneic mice.

### DETAILED DESCRIPTION OF THE DISCLOSURE

Described herein are methods for treating proliferative diseases, in particular, methods for treating cancer. The methods comprise administering a combination of a tyrosine kinase inhibitor, or a pharmaceutically acceptable salt thereof, and an immunotherapeutic agent, or a pharmaceutically acceptable salt thereof. Also described, are methods for enhancing the efficacy of a tyrosine kinase inhibitor to treat cancer, comprising administering the tyrosine kinase inhibitor in combination with an immunotherapeutic agent. Also described, are methods for enhancing the efficacy of an immunotherapeutic agent to treat cancer, comprising administering the immunotherapeutic agent in combination with a tyrosine kinase inhibitor. The combination of the tyrosine kinase inhibitor, or a pharmaceutically acceptable salt thereof with the immunotherapeutic agent, or a pharmaceutically acceptable salt thereof, enhances the efficacy of either of the agents alone, to treat cancer. In some embodiments, the combination of the tyrosine kinase inhibitor, or a pharmaceutically acceptable salt thereof with the immunotherapeutic agent, or a pharmaceutically acceptable salt thereof, act synergistically to treat cancer. Also described, are methods for inhibiting a cancer associated tumor growth comprising administering a combination of a tyrosine kinase inhibitor, or a pharmaceutically acceptable salt thereof, and an immunotherapeutic agent, or a pharmaceutically acceptable salt thereof.

In some embodiments, the combination treatments of apatinib mesylate and immunotherapeutic agent synergistically suppress tumor growth.

In one aspect, the present disclosure relates to the use of apatinib mesylate combining with current immunotherapeutic agents to treat tumors.

### Certain Terminologies

As used herein, the term “combination therapy” refers to a combination of therapeutically active agents (apatinib and an immunotherapeutic agent) encompassed in single or multiple compositions. The therapeutically active agents may be administered together at the same time to a patient in need thereof or separately (each or in any combinations thereof) in any sequential manner prescribed by a medical care taker.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of a disorder of uncontrolled cellular proliferation, such as cancer.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, zebra fish etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder of uncontrolled cellular proliferation” means having been subjected to a physical examination by a person of skill, for example, a physician, and found to have a condition that can be diagnosed or treated by a compound or composition that can inhibit uncontrolled cellular proliferation. Such a diagnosis can be in reference to a disorder, such as a disorder of uncontrolled cellular proliferation, cancer and the like, as discussed herein.

As used herein, the phrase “identified to be in need of treatment for a disorder,” or the like, refers to selection of a subject based upon need for treatment of the disorder. For example, a subject can be identified as having a need for treatment of a disorder based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, intraurethral administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “therapeutically effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “pharmaceutically acceptable carrier” relates to pharmaceutically-acceptable, nontoxic carriers or diluents, which are defined as vehicles commonly used to formulate pharmaceutical compositions for animal or human administration. Such carriers may include, however not limited to, buffering agents, solubilizing agents, stabilizing agents or taste additives.

The term “immunotherapy” refers to any treatment of a disease by inducing, enhancing, or suppressing an immune response. Immunotherapies designed to elicit or amplify an immune response are classified as activation immunotherapies, while immunotherapies that reduce or suppress an immune response are classified as suppression immunotherapies.

Unless defined otherwise, all technical and scientific terms used herein have the same meaning as is commonly understood by one of skill in the art to which the claimed subject matter belongs. It is to be understood that the detailed description are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the disclosure may be described in the context of a single embodiment, the features may also be provided separately or in any suitable combination. Conversely, although the disclosure may be described herein in the context of separate embodiments for clarity, the disclosure may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the disclosures.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error. Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

The section headings used herein are for organizational purposes only and are not to be construed as limiting the subject matter described.

The present disclosure is generally directed to therapies that are useful to alleviate, abate or eliminate one or more diseases or conditions in a subject in need thereof, as further described herein. In particular, described herein are methods for treating diseases, where the methods comprise administering a combination of two or more therapies, in particular a combination comprising a tyrosine kinase inhibitor, or a pharmaceutically acceptable salt thereof and an immunotherapeutic agent or a pharmaceutically acceptable salt thereof.

The singular forms “a,” “an,” and, “the” include plural referents unless the context clearly dictates otherwise. Thus, for example, reference to “the surfactant” includes reference to one or more specific surfactants, reference to “an antioxidant” includes reference to one or more of such additives.

### Tyrosine Kinase Inhibitors

Various embodiments described herein are directed to methods for treating diseases, comprising administering a tyrosine kinase inhibitor, or a pharmaceutically acceptable thereof.

Tyrosine kinases (or protein tyrosine kinases, PTK) are enzymes that activate and regulate cell proliferation signaling pathways. Overexpression of the PTK gene enhances PTK activity, altering its downstream signaling pathways, causing cell proliferation disorders, and eventually leading to tumor formation. Protein tyrosine kinases occur in two forms—Receptor PTK (RTK) and Non-receptor PTK (NRTK). Receptor RTKs include the epidermal growth factor receptor (EGFR), platelet-derived growth factor receptor (PDGFR), vascular endothelial growth factor receptor (VEGFR) and insulin receptor (InsR) families. They comprise an extracellular binding domain, a transmembrane region, and an intracellular kinase domain that selectively binds to and phosphorylates the substrate. RTK can bind to ligands and phosphorylate tyrosine residues of target proteins and transmit information to activate a series of biochemical reactions; or different information combined to cause a comprehensive cellular response (such as cell proliferation). Clinical studies in cancer have shown that these receptors and their ligands play a significant role in tumor formation and/or growth. Many cancers have over-expressed growth factors that cause excessive tyrosine phosphorylation signal into cells.

VEGFR family members include VEGFR1, VEGFR2 and VEGFR3. The family of receptors has seven immunoglobulin like domains and a hydrophilic insert sequence in the intracellular tyrosine kinase region. VEGF plays an important role in the proliferation, migration, and vascularization of endothelial cells as the most powerful vascular penetrant and endothelium-specific mitotic source. There is significant positive correlation between the VEGFR expression level and the degree of vascularization and malignancy of tumor tissue. Among them, VEGFR2 is the most important in mediating the biological effect of VEGF, which is closely related to cell chemotaxis and cell division.

Tyrosine kinase inhibitors block the action of tyrosine kinase enzymes. Development of kinase inhibitors for the treatment of cancer has proven successful, with protein kinases now the second most targeted group of drug targets. Over thirty kinase inhibitors have received FDA approval; over 150 are in clinical trials, and many more are in preclinical development. A recent review of kinase targeted cancer therapies (see “Kinase-targeted cancer therapies: progress, challenges and future directions”, Bhullar, et al, Mol Cancer, 2018, 17, 48) provided FDA-approved kinase inhibitors and their drug targets, summarized in Table 1.

TABLE 1

| | Protein | |
|---|---|---|
| Drug target | substrate | Drug |
| VEGFR | Tyrosine | Axitinib, Lenvatinib, Nintedanib, Regorafenib, |
| family | | Pazopanib, Sorafenib, Sunitinib |
| ALK | Tyrosine | Crizotinib, Ceritinib, Alectinib, Brigatinib |
| BCR-Ab1 | Tyrosine | Bosutinib, Dasatinib, Imatinib, Nilotinib, |
| | | Ponatinib |
| BTK | Tyrosine | Ibrutinib |
| c-Met | Tyrosine | Crizotinib, Cabozantinib |
| EGFR | Tyrosine | Gefitinib, Erlotinib, Lapatinib, Vandetanib, |
| family | | Afatinib, Osimertinib |
| JAK family | Tyrosine | Ruxolitinib, Tofacitinib |
| PDGFR | Tyrosine | Axitinib, Gefitinib, Imatinib, Lenvatinib, |
| α/β | | Nintedanib, Pazopanib, Regorafenib, Sorafenib, |
| | | Sunitinib |
| RET | Tyrosine | Vandetanib |
| Src family | Tyrosine | Bosutinib, Dasatinib, Ponatinib, Vandetanib |
| MEK1/2 | Dual | Trametinib |
| B-Raf | Ser/thre | Vemurafenib, Dabrafenib |
| CDK family | Ser/thre | Palbociclib, Sorafenib, Ribociclib |

However, many factors complicate and impede the clinical efficacy of these drugs. Specific tumor genetics, tumor microenvironment, drug resistance, and pharmacogenomics determine how useful a compound will be in the treatment of a given cancer and these factors are difficult, if not impossible, to predict. For example, some observed safety issues for approved drugs are presented in Table 2.

TABLE 2

| Safety Profile | Bosutinib | Dasatinib | Erlotinib | Gefitinib | Imatinib | Lapatinib | Nilotinib | Pazopanib | Ponatinib | Sorafenib | Sunitinib |
|---|---|---|---|---|---|---|---|---|---|---|---|
| GI | C | C | C | C | C | C | R | C | C | R | C |
| Renal | C | R | C | C | R | | R | C | | R | C |
| Musculoskeletal/ | C | R | | | C | C | R | C | C | R | C |
| bone | | | | | | | | | | | |
| Blood/lymph | C | C | R | | C | | C | C | C | C | C |
| system | | | | | | | | | | | |
| Vascular | | C | | C | R | C | R | C | C | C | C |
| Skin | C | C | C | C | C | C | C | C | C | C | C |
| CMR | | C | C | C | C | C | C | C | | C | C |
| Central nervous | | R | R | | R | R | R | | | R | R |
| system | | | | | | | | | | | |
| Nerve | C | C | C | | C | C | C | C | C | C | C |
| Eye | | C | C | C | C | | C | C | C | | C |
| Heart | C | C | | | R | R | C | R | C | R | R |
| Lung airways | C | C | C | C | C | C | C | C | C | R | C |
| Thyroid | | | | | R | | | C | | | C |
| Liver/Bile | C | R | R | C | C | C | C | C | C | R | R |

C = common or very common; R = rare
CMR, carcinogenic, mutagenic and toxic for reproductive system

Many more tyrosine kinase inhibitors exist, including but not limited, to afatinib, alectinib, apatinib, axitinib, bosutinib, brigatinib, cabozantinib, canertinib, ceritinib, crizotinib, dabrafenib, danusertib, dasatinib, erlotinib, gefitinib, ibrutinib, imatinib, lapatinib, lenvatinib, linifanib, masitinib, neratinib, nilotinib, nintedanib, orantinib, osimertinib, palbociclib, pazopanib, ponatinib, quizartinib, rebastinib, regorafenib, ribociclib, rivoceranib, ruxolitinib, sapitinib, semaxinib, sorafenib, sunitinib, tandutinib, tofacitinib, trametinib, vandetanib, vatalanib, vemurafenib and vismodegib.

VEGF/VEGFR inhibitors include but are not limited to aflibercept, allantoin, bevacizumab, lenvatinib, pazopanib, pegaptanib, ramucirumab, ranibizumab, sunitinib, tivozanib, and vandetanib.

### Rivoceranib (Apatinib)

Rivoceranib (chemical name N-[4-(1-cyanocyclopentyl)phenyl]-2-{[(pyridin-4-yl)methyl]amino} pyridine-3-carboxamide, also known as YN968D1, developed in China as apatinib and marketed as Aitan®) is an orally administered small molecule tyrosine kinase inhibitor. It selectively inhibits vascular endothelial growth factor receptor (VEGFR)-2 leading to blockage of tumor vascular angiogenesis, diminishes survival of existing blood vessels, and retards growth of tumors. Proliferation of endothelial cells is targeted directly, and inhibition of the release of proangiogenic growth factors by cancer or stromal cells is targeted indirectly.

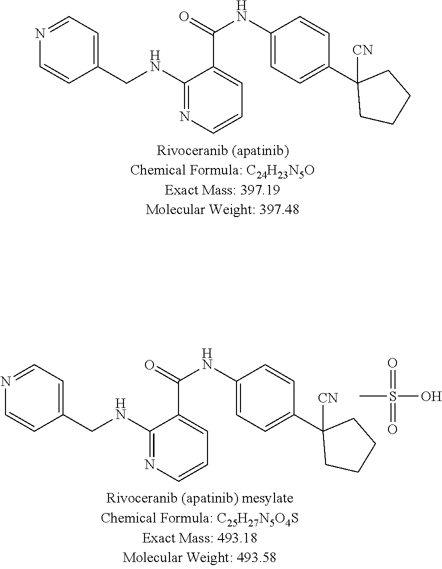

Nonclinical studies completed with rivoceranib demonstrated:
Rivoceranib selectively binds to VEGFR-2 and is efficacious in various tumor bearing animal models.
There are minimal side effects in animals at efficacious doses of rivoceranib.
Rivoceranib is high binding compound with over 97% binding to plasma albumin.
Rivoceranib is generally poorly absorbed.
Steady state levels of Rivoceranib were achieved in approximately 7 days; further accumulation was not evident in studies up to 28-days.

Rivoceranib has been clinically tested in over 1,000 patients and has demonstrated efficacy in numerous cancers including gastric cancer, colorectal cancer (CRC), hepatocellular carcinoma (HCC), non-small-cell lung cancer (NSCLC), esophageal cancer, thyroid cancer, mesothelioma, and neuroendocrine tumors. Several clinical studies of rivoceranib have been completed and are briefly described below.

A Phase 1 study (46 patients) revealed a once daily dose-limiting toxicity of 805 mg rivoceranib (1000 mg rivoceranib mesylate) and a maximum tolerated dose of 685 mg (850 mg mesylate salt). Partial response was noted in 7 patients (19%), stable disease in 24 patients (65%), and a disease control rate of 84% at 8 weeks.

A Phase ½a dose escalation and PK study provided a recommended Phase 2a dose of 685 mg (850 mg mesylate salt), where thirty patients then received up to 685 mg rivoceranib (850 mg mesylate salt) in 28-Day cycles (2 cycles). 5 deaths were reported during the study, though a clinical disease control rate was achieved for 93% of patients (n=28 evaluable patients).

A Phase 1 study to evaluate the PK of rivoceranib with and without food for two doses of rivoceranib mesylate (100 mg and 250 mg rivoceranib mesylate, corresponding to 81 mg and 201 mg freebase, respectively) administered in healthy volunteers. Food effects on the bioavailability of the 81 mg rivoceranib dose were minimal, while more pronounced (20-30% increase in bioavailability) for the 201 mg dose.

A Phase 1 study was conducted to evaluate a single dose of rivoceranib mesylate in healthy male patients of Caucasian, Japanese and Chinese origin. The results showed that Cmax and AUC0-∞ in Chinese and Japanese subjects were slightly higher compared to Caucasian subjects, while t_{1/2} values were similar (7.5-8 hours) amongst the three groups.

A Phase 2 study of patients with advanced or metastatic gastric cancer after failure of 2 lines of chemotherapy was completed, where rivoceranib was dosed daily at 685 mg (850 mg rivoceranib mesylate). This study provided placebo-controlled evidence that rivoceranib has significant activity against gastric cancer with a manageable safety profile. In a follow-up to this study, a Phase 3 multi-center, randomized, double-blind, and placebo-controlled study was conducted. In this study, efficacy, median overall survival (OS) and median progression-free survival (PFS) were prolonged in the rivoceranib group compared to placebo. The recommended dose for clinical use was 685 mg rivoceranib (850 mg mesylate salt) once daily. Treatment with rivoceranib was generally well tolerated with most of the adverse reactions manageable by dose interruptions or reductions. Grade ¾ adverse reactions that occurred in more than 2% of patients were hypertension, HFS, proteinuria, fatigue, anorexia, and elevated aminotransferase.

In December 2014, Rivoceranib received approval in China, for treatment of advanced gastric cancer, and has received orphan medicinal product designation for the treatment of gastric cancer from Europe, the FDA, and the MFDS in South Korea. However, according to a recent review (see L. J. Scott, “Apatinib: A Review in Advanced Gastric Cancer and Other Advanced Cancers,” Drugs, 2018, 78(7), 747-758), “further clinical experience and long-term pharmacovigilance data are required to more definitively establish the efficacy and safety profile of apatinib, including its use in combination with other chemotherapy agents and its role in the management of other types of advanced or metastatic solid tumors”.

In some embodiments, the methods for treating diseases comprise administering a combination of two or more therapies, wherein one of the therapies is a tyrosine kinase inhibitor, or a pharmaceutically acceptable salt thereof.

In some embodiments, the tyrosine kinase inhibitor is administered in the form of a free base. In some embodiments, the tyrosine kinase inhibitor is administered in the form of a pharmaceutically acceptable salt. As used herein, a pharmaceutically acceptable salt includes, but is not limited to, metal salts, such as sodium salts, potassium salts, and lithium salts; alkaline earth metals, such as calcium salts, magnesium salts, and the like; organic amine salts, such as triethylamine salts, pyridine salts, picoline salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparginate salts, glutamate salts, and the like. Pharmaceutically acceptable salts also include bitartrate, bitartrate hydrate, hydrochloride, p-toluenesulfonate, phosphate, sulfate, trifluoroacetate, bitartrate hemipentahydrate, pentafluoropropionate, hydrobromide, mucate, oleate, phosphate dibasic, phosphate monobasic, acetate trihydrate, bis(heptafuorobutyrate), bis(pentafluoropropionate), bis(pyridine carboxylate), bis(trifluoroacetate), chlorhydrate, and sulfate pentahydrate. Other representative pharmaceutically acceptable salts include, e.g., water-soluble and water-insoluble salts, such as the acetate, amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A hydrate is another example of a pharmaceutically acceptable salt.

In some embodiments, the tyrosine kinase inhibitor selective vascular endothelial growth factor receptor-2 (VEGFR-2) inhibitor. In some embodiments, the tyrosine kinase inhibitor isafatinib, alectinib, apatinib, axitinib, bosutinib, brigatinib, canertinib, crizotinib, ceritinib, dasatinib, danusertib, dabrafenib, erlotinib, gefitinib, ibrutinib, imatinib, lapatinib, lenvatinib, neratinib, nilotinib, nintedanib, osimertinib, palbociclib, pazopanib, pegaptanib, ponatinib, rebastinib, regorafenib, ribociclib, rivoceranib, ruxolitinib, semaxinib, sorafenib, sunitinib, tivozanib, trametinib, tofacitinib, vandetanib, vatalanib, vemurafenib or vismodegib. In some embodiments, the tyrosine kinase inhibitor is rivoceranib. In some embodiments, the tyrosine kinase inhibitor is a pharmaceutically acceptable salt of rivoceranib. In some embodiments, the tyrosine kinase inhibitor is rivoceranib mesylate.

In some embodiments, the tyrosine kinase inhibitor is administered in an amount of at least 10 mg. In some embodiments, the tyrosine kinase inhibitor is administered in an amount of at least 50 mg. In some embodiments, the tyrosine kinase inhibitor is administered in an amount of at least 100 mg. In some embodiments, the tyrosine kinase inhibitor is administered in an amount of at least 150 mg. In some embodiments, the tyrosine kinase inhibitor is administered in an amount of at least 200 mg. In some embodiments, the tyrosine kinase inhibitor is administered in an amount of at least 225 mg. In some embodiments, the tyrosine kinase inhibitor is administered in an amount of at least 250 mg. In some embodiments, the tyrosine kinase inhibitor is administered in an amount of at least 275 mg. In some embodiments, the tyrosine kinase inhibitor is administered in an amount of at least 300 mg. In some embodiments, the tyrosine kinase inhibitor is administered in an amount of at least 310 mg. In some embodiments, the tyrosine kinase inhibitor is administered in an amount of at least 320 mg. In some embodiments, the tyrosine kinase inhibitor is administered in an amount of at least 325 mg. In some embodiments, the tyrosine kinase inhibitor is administered in an amount of at least 330 mg. In some embodiments, the tyrosine kinase inhibitor is administered in an amount of at least 340 mg. In some embodiments, the tyrosine kinase inhibitor is administered in an amount of at least 350 mg. In some embodiments, the tyrosine kinase inhibitor is administered in an amount of at least 360 mg. In some embodiments, the tyrosine kinase inhibitor is administered in an amount of at least 370 mg. In some embodiments, the tyrosine kinase inhibitor is administered in an amount of at least 375 mg. In some embodiments, the tyrosine kinase inhibitor is administered in an amount of at least 380 mg. In some embodiments, the tyrosine kinase inhibitor is administered in an amount of at least 400 mg. In some embodiments, the tyrosine kinase inhibitor is administered in an amount of at least 410 mg. In some embodiments, the tyrosine kinase inhibitor is administered in an amount of at least 420 mg. In some embodiments, the tyrosine kinase inhibitor is administered in an amount of at least 425 mg. In some embodiments, the tyrosine kinase inhibitor is administered in an amount of at least 430 mg. In some embodiments, the tyrosine kinase inhibitor is administered in an amount of at least 440 mg. In some embodiments, the tyrosine kinase inhibitor is administered in an amount of at least 450 mg. In some embodiments, the tyrosine kinase inhibitor is administered in an amount of at least 460 mg. In some embodiments, the tyrosine kinase inhibitor is administered in an amount of at least 470 mg. In some embodiments, the tyrosine kinase inhibitor is administered in an amount of at least 475 mg. In some embodiments, the tyrosine kinase inhibitor is administered in an amount of at least 480 mg. In some embodiments, the tyrosine kinase inhibitor is administered in an amount of at least 490 mg. In some embodiments, the tyrosine kinase inhibitor is administered in an amount of at least 500 mg. In some embodiments, the tyrosine kinase inhibitor is administered in an amount of at least 525 mg. In some embodiments, the tyrosine kinase inhibitor is administered in an amount of at least 550 mg. In some embodiments, the tyrosine kinase inhibitor is administered in an amount of at least 575 mg. In some embodiments, the tyrosine kinase inhibitor is administered in an amount of at least 600 mg. In some embodiments, the tyrosine kinase inhibitor is administered in an amount of at least 625 mg. In some embodiments, the tyrosine kinase inhibitor is administered in an amount of at least 650 mg. In some embodiments, the tyrosine kinase inhibitor is administered in an amount of at least 675 mg. In some embodiments, the tyrosine kinase inhibitor is administered in an amount of at least 700 mg. In some embodiments, the tyrosine kinase inhibitor is rivoceranib. In some embodiments, the tyrosine kinase inhibitor is a pharmaceutically acceptable salt of rivoceranib. In some embodiments, the tyrosine kinase inhibitor is rivoceranib mesylate.

In some embodiments, the tyrosine kinase inhibitor is administered in an amount of about 10 mg. In some embodiments, the tyrosine kinase inhibitor is administered in an amount of about 50 mg. In some embodiments, the tyrosine kinase inhibitor is administered in an amount of about 100 mg. In some embodiments, the tyrosine kinase inhibitor is administered in an amount of about 150 mg. In some embodiments, the tyrosine kinase inhibitor is administered in an amount of about 200 mg. In some embodiments, the tyrosine kinase inhibitor is administered in an amount of about 225 mg. In some embodiments, the tyrosine kinase inhibitor is administered in an amount of about 250 mg. In some embodiments, the tyrosine kinase inhibitor is administered in an amount of about 275 mg. In some embodiments, the tyrosine kinase inhibitor is administered in an amount of about 300 mg. In some embodiments, the tyrosine kinase inhibitor is administered in an amount of about 310 mg. In some embodiments, the tyrosine kinase inhibitor is administered in an amount of about 320 mg. In some embodiments, the tyrosine kinase inhibitor is administered in an amount of about 325 mg. In some embodiments, the tyrosine kinase inhibitor is administered in an amount of about 330 mg. In some embodiments, the tyrosine kinase inhibitor is administered in an amount of about 340 mg. In some embodiments, the tyrosine kinase inhibitor is administered in an amount of about 350 mg. In some embodiments, the tyrosine kinase inhibitor is administered in an amount of about 360 mg. In some embodiments, the tyrosine kinase inhibitor is administered in an amount of about 370 mg. In some embodiments, the tyrosine kinase inhibitor is administered in an amount of about 375 mg. In some embodiments, the tyrosine kinase inhibitor is administered in an amount of about 380 mg. In some embodiments, the tyrosine kinase inhibitor is administered in an amount of about 400 mg. In some embodiments, the tyrosine kinase inhibitor is administered in an amount of about 410 mg. In some embodiments, the tyrosine kinase inhibitor is administered in an amount of about 420 mg. In some embodiments, the tyrosine kinase inhibitor is administered in an amount of about 425 mg. In some embodiments, the tyrosine kinase inhibitor is administered in an amount of about 430 mg. In some embodiments, the tyrosine kinase inhibitor is administered in an amount of about 440 mg. In some embodiments, the tyrosine kinase inhibitor is administered in an amount of about 450 mg. In some embodiments, the tyrosine kinase inhibitor is administered in an amount of about 460 mg. In some embodiments, the tyrosine kinase inhibitor is administered in an amount of about 470 mg. In some embodiments, the tyrosine kinase inhibitor is administered in an amount of about 475 mg. In some embodiments, the tyrosine kinase inhibitor is administered in an amount of about 480 mg. In some embodiments, the tyrosine kinase inhibitor is administered in an amount of about 490 mg. In some embodiments, the tyrosine kinase inhibitor is administered in an amount of about 500 mg. In some embodiments, the tyrosine kinase inhibitor is administered in an amount of about 525 mg. In some embodiments, the tyrosine kinase inhibitor is administered in an amount of about 550 mg. In some embodiments, the tyrosine kinase inhibitor is administered in an amount of about 575 mg. In some embodiments, the tyrosine kinase inhibitor is administered in an amount of about 600 mg. In some embodiments, the tyrosine kinase inhibitor is administered in an amount of about 625 mg. In some embodiments, the tyrosine kinase inhibitor is administered in an amount of about 650 mg. In some embodiments, the tyrosine kinase inhibitor is administered in an amount of about 675 mg. In some embodiments, the tyrosine kinase inhibitor is administered in an amount of about 700 mg. In some embodiments, the tyrosine kinase inhibitor is rivoceranib. In some embodiments, the tyrosine kinase inhibitor is a pharmaceutically acceptable salt of rivoceranib. In some embodiments, the tyrosine kinase inhibitor is rivoceranib mesylate.

In some embodiments, rivoceranib is administered in an amount of about 10 mg. In some embodiments, rivoceranib is administered in an amount of about 50 mg. In some embodiments, rivoceranib is administered in an amount of about 100 mg. In some embodiments, rivoceranib is administered in an amount of about 105 mg. In some embodiments, rivoceranib is administered in an amount of about 110 mg. In some embodiments, rivoceranib is administered in an amount of about 115 mg. In some embodiments, rivoceranib is administered in an amount of about 120 mg. In some embodiments, rivoceranib is administered in an amount of about 125 mg. In some embodiments, rivoceranib is administered in an amount of about 130 mg. In some embodiments, rivoceranib is administered in an amount of about 135 mg. In some embodiments, rivoceranib is administered in an amount of about 140 mg. In some embodiments, rivoceranib is administered in an amount of about 145 mg. In some embodiments, rivoceranib is administered in an amount of about 150 mg. In some embodiments, rivoceranib is administered in an amount of about 200 mg. In some embodiments, rivoceranib is administered in an amount of about 225 mg. In some embodiments, rivoceranib is administered in an amount of about 250 mg. In some embodiments, rivoceranib is administered in an amount of about 275 mg. In some embodiments, rivoceranib is administered in an amount of about 300 mg. In some embodiments, rivoceranib is administered in an amount of about 310 mg. In some embodiments, rivoceranib is administered in an amount of about 320 mg. In some embodiments, rivoceranib is administered in an amount of about 325 mg. In some embodiments, rivoceranib is administered in an amount of about 330 mg. In some embodiments, rivoceranib is administered in an amount of about 340 mg. In some embodiments, rivoceranib is administered in an amount of about 350 mg. In some embodiments, rivoceranib is administered in an amount of about 360 mg. In some embodiments, rivoceranib is administered in an amount of about 370 mg. In some embodiments, rivoceranib is administered in an amount of about 375 mg. In some embodiments, rivoceranib is administered in an amount of about 380 mg. In some embodiments, rivoceranib is administered in an amount of about 400 mg. In some embodiments, rivoceranib is administered in an amount of about 410 mg. In some embodiments, rivoceranib is administered in an amount of about 420 mg. In some embodiments, rivoceranib is administered in an amount of about 425 mg. In some embodiments, rivoceranib is administered in an amount of about 430 mg. In some embodiments, rivoceranib is administered in an amount of about 440 mg. In some embodiments, rivoceranib is administered in an amount of about 450 mg. In some embodiments, rivoceranib is administered in an amount of about 460 mg. In some embodiments, rivoceranib is administered in an amount of about 470 mg. In some embodiments, rivoceranib is administered in an amount of about 475 mg. In some embodiments, rivoceranib is administered in an amount of about 480 mg. In some embodiments, rivoceranib is administered in an amount of about 490 mg. In some embodiments, rivoceranib is administered in an amount of about 500 mg. In some embodiments, rivoceranib is administered in an amount of about 525 mg. In some embodiments, rivoceranib is administered in an amount of about 550 mg. In some embodiments, rivoceranib is administered in an amount of about 575 mg. In some embodiments, rivoceranib is administered in an amount of about 600 mg. In some embodiments, rivoceranib is administered in an amount of about 625 mg. In some embodiments, rivoceranib is administered in an amount of about 650 mg. In some embodiments, rivoceranib is administered in an amount of about 675 mg. In some embodiments, rivoceranib is administered in an amount of about 700 mg.

In some embodiments, rivoceranib mesylate is administered in an amount of about 10 mg. In some embodiments, rivoceranib mesylate is administered in an amount of about 50 mg. In some embodiments, rivoceranib mesylate is administered in an amount of about 100 mg. In some embodiments, rivoceranib mesylate is administered in an amount of about 150 mg. In some embodiments, rivoceranib mesylate is administered in an amount of about 200 mg. In some embodiments, rivoceranib mesylate is administered in an amount of about 225 mg. In some embodiments, rivoceranib mesylate is administered in an amount of about 250 mg. In some embodiments, rivoceranib mesylate is administered in an amount of about 275 mg. In some embodiments, rivoceranib mesylate is administered in an amount of about 300 mg. In some embodiments, rivoceranib mesylate is administered in an amount of about 310 mg. In some embodiments, rivoceranib mesylate is administered in an amount of about 320 mg. In some embodiments, rivoceranib mesylate is administered in an amount of about 325 mg. In some embodiments, rivoceranib mesylate is administered in an amount of about 330 mg. In some embodiments, rivoceranib mesylate is administered in an amount of about 340 mg. In some embodiments, rivoceranib mesylate is administered in an amount of about 350 mg. In some embodiments, rivoceranib mesylate is administered in an amount of about 360 mg. In some embodiments, rivoceranib mesylate is administered in an amount of about 370 mg. In some embodiments, rivoceranib mesylate is administered in an amount of about 375 mg. In some embodiments, rivoceranib mesylate is administered in an amount of about 380 mg. In some embodiments, rivoceranib mesylate is administered in an amount of about 400 mg. In some embodiments, rivoceranib mesylate is administered in an amount of about 410 mg. In some embodiments, rivoceranib mesylate is administered in an amount of about 420 mg. In some embodiments, rivoceranib mesylate is administered in an amount of about 425 mg. In some embodiments, rivoceranib mesylate is administered in an amount of about 430 mg. In some embodiments, rivoceranib mesylate is administered in an amount of about 440 mg. In some embodiments, rivoceranib mesylate is administered in an amount of about 450 mg. In some embodiments, rivoceranib mesylate is administered in an amount of about 460 mg. In some embodiments, rivoceranib mesylate is administered in an amount of about 470 mg. In some embodiments, rivoceranib mesylate is administered in an amount of about 475 mg. In some embodiments, rivoceranib mesylate is administered in an amount of about 480 mg. In some embodiments, rivoceranib mesylate is administered in an amount of about 490 mg. In some embodiments, rivoceranib mesylate is administered in an amount of about 500 mg. In some embodiments, rivoceranib mesylate is administered in an amount of about 525 mg. In some embodiments, rivoceranib mesylate is administered in an amount of about 550 mg. In some embodiments, rivoceranib mesylate is administered in an amount of about 575 mg. In some embodiments, rivoceranib mesylate is administered in an amount of about 600 mg. In some embodiments, rivoceranib mesylate is administered in an amount of about 625 mg. In some embodiments, rivoceranib mesylate is administered in an amount of about 650 mg. In some embodiments, rivoceranib mesylate is administered in an amount of about 675 mg. In some embodiments, rivoceranib mesylate is administered in an amount of about 700 mg.

In some embodiments, the tyrosine kinase inhibitor is administered in an amount of no more than 10 mg. In some embodiments, the tyrosine kinase inhibitor is administered in an amount of no more than 50 mg. In some embodiments, the tyrosine kinase inhibitor is administered in an amount of no more than 100 mg. In some embodiments, the tyrosine kinase inhibitor is administered in an amount of no more than 150 mg. In some embodiments, the tyrosine kinase inhibitor is administered in an amount of no more than 200 mg. In some embodiments, the tyrosine kinase inhibitor is administered in an amount of no more than 225 mg. In some embodiments, the tyrosine kinase inhibitor is administered in an amount of no more than 250 mg. In some embodiments, the tyrosine kinase inhibitor is administered in an amount of no more than 275 mg. In some embodiments, the tyrosine kinase inhibitor is administered in an amount of no more than 300 mg. In some embodiments, the tyrosine kinase inhibitor is administered in an amount of no more than 310 mg. In some embodiments, the tyrosine kinase inhibitor is administered in an amount of no more than 320 mg. In some embodiments, the tyrosine kinase inhibitor is administered in an amount of no more than 325 mg. In some embodiments, the tyrosine kinase inhibitor is administered in an amount of no more than 330 mg. In some embodiments, the tyrosine kinase inhibitor is administered in an amount of no more than 340 mg. In some embodiments, the tyrosine kinase inhibitor is administered in an amount of no more than 350 mg. In some embodiments, the tyrosine kinase inhibitor is administered in an amount of no more than 360 mg. In some embodiments, the tyrosine kinase inhibitor is administered in an amount of no more than 370 mg. In some embodiments, the tyrosine kinase inhibitor is administered in an amount of no more than 375 mg. In some embodiments, the tyrosine kinase inhibitor is administered in an amount of no more than 380 mg. In some embodiments, the tyrosine kinase inhibitor is administered in an amount of no more than 400 mg. In some embodiments, the tyrosine kinase inhibitor is administered in an amount of no more than 410 mg. In some embodiments, the tyrosine kinase inhibitor is administered in an amount of no more than 420 mg. In some embodiments, the tyrosine kinase inhibitor is administered in an amount of no more than 425 mg. In some embodiments, the tyrosine kinase inhibitor is administered in an amount of no more than 430 mg. In some embodiments, the tyrosine kinase inhibitor is administered in an amount of no more than 440 mg. In some embodiments, the tyrosine kinase inhibitor is administered in an amount of no more than 450 mg. In some embodiments, the tyrosine kinase inhibitor is administered in an amount of no more than 460 mg. In some embodiments, the tyrosine kinase inhibitor is administered in an amount of no more than 470 mg. In some embodiments, the tyrosine kinase inhibitor is administered in an amount of no more than 475 mg. In some embodiments, the tyrosine kinase inhibitor is administered in an amount of no more than 480 mg. In some embodiments, the tyrosine kinase inhibitor is administered in an amount of no more than 490 mg. In some embodiments, the tyrosine kinase inhibitor is administered in an amount of no more than 500 mg. In some embodiments, the tyrosine kinase inhibitor is administered in an amount of no more than 525 mg. In some embodiments, the tyrosine kinase inhibitor is administered in an amount of no more than 550 mg. In some embodiments, the tyrosine kinase inhibitor is administered in an amount of no more than 575 mg. In some embodiments, the tyrosine kinase inhibitor is administered in an amount of no more than 600 mg. In some embodiments, the tyrosine kinase inhibitor is administered in an amount of no more than 625 mg. In some embodiments, the tyrosine kinase inhibitor is administered in an amount of no more than 650 mg. In some embodiments, the tyrosine kinase inhibitor is administered in an amount of no more than 675 mg. In some embodiments, the tyrosine kinase inhibitor is administered in an amount of no more than 700 mg. In some embodiments, the tyrosine kinase inhibitor is rivoceranib. In some embodiments, the tyrosine kinase inhibitor is a pharmaceutically acceptable salt of rivoceranib. In some embodiments, the tyrosine kinase inhibitor is rivoceranib mesylate.

In some embodiments, the tyrosine kinase inhibitor is administered in an amount of from 100 mg to 900 mg. In some embodiments, the tyrosine kinase inhibitor is administered in an amount of from 150 mg to 850 mg. In some embodiments, the tyrosine kinase inhibitor is administered in an amount of from 175 mg to 825 mg. In some embodiments, the tyrosine kinase inhibitor is administered in an amount of from 200 mg to 800 mg. In some embodiments, the tyrosine kinase inhibitor is administered in an amount of from 225 mg to 775 mg. In some embodiments, the tyrosine kinase inhibitor is administered in an amount of from 250 mg to 750 mg. In some embodiments, the tyrosine kinase inhibitor is administered in an amount of from 250 mg to 725 mg. In some embodiments, the tyrosine kinase inhibitor is administered in an amount of from 250 mg to 700 mg. In some embodiments, the tyrosine kinase inhibitor is administered in an amount of from 250 mg to 675 mg. In some embodiments, the tyrosine kinase inhibitor is administered in an amount of from 250 mg to 650 mg. In some embodiments, the tyrosine kinase inhibitor is administered in an amount of from 250 mg to 600 mg. In some embodiments, the tyrosine kinase inhibitor is administered in an amount of from 250 mg to 550 mg. In some embodiments, the tyrosine kinase inhibitor is administered in an amount of from 250 mg to 500 mg. In some embodiments, the tyrosine kinase inhibitor is administered in an amount of from 250 mg to 450 mg. In some embodiments, the tyrosine kinase inhibitor is administered in an amount of from 275 mg to 425 mg. In some embodiments, the tyrosine kinase inhibitor is administered in an amount of from 300 mg to 600 mg. In some embodiments, the tyrosine kinase inhibitor is administered in an amount of from 300 mg to 550 mg. In some embodiments, the tyrosine kinase inhibitor is administered in an amount of from 300 mg to 500 mg. In some embodiments, the tyrosine kinase inhibitor is administered in an amount of from 300 mg to 450 mg. In some embodiments, the tyrosine kinase inhibitor is administered in an amount of from 300 mg to 400 mg. In some embodiments, the tyrosine kinase inhibitor is administered in an amount of from 350 mg to 450 mg. In some embodiments, the tyrosine kinase inhibitor is rivoceranib. In some embodiments, the tyrosine kinase inhibitor is a pharmaceutically acceptable salt of rivoceranib. In some embodiments, the tyrosine kinase inhibitor is rivoceranib mesylate

In some embodiments, rivoceranib is administered in an amount of less than 685 mg.

In some embodiments, rivoceranib mesylate is administered in an amount of less than 685 mg.

In some embodiments, the total daily dose of the tyrosine kinase inhibitor is about 10 mg. In some embodiments, the total daily dose of the tyrosine kinase inhibitor is about 50 mg. In some embodiments, the total daily dose of the tyrosine kinase inhibitor is about 100 mg. In some embodiments, the total daily dose of the tyrosine kinase inhibitor is about 150 mg. In some embodiments, the total daily dose of the tyrosine kinase inhibitor is about 200 mg. In some embodiments, the total daily dose of the tyrosine kinase inhibitor is about 225 mg. In some embodiments, the total daily dose of the tyrosine kinase inhibitor is about 250 mg. In some embodiments, the total daily dose of the tyrosine kinase inhibitor is about 275 mg. In some embodiments, the total daily dose of the tyrosine kinase inhibitor is about 300 mg. In some embodiments, the total daily dose of the tyrosine kinase inhibitor is about 310 mg. In some embodiments, the total daily dose of the tyrosine kinase inhibitor is about 320 mg. In some embodiments, the total daily dose of the tyrosine kinase inhibitor is about 325 mg. In some embodiments, the total daily dose of the tyrosine kinase inhibitor is about 330 mg. In some embodiments, the total daily dose of the tyrosine kinase inhibitor is about 340 mg. In some embodiments, the total daily dose of the tyrosine kinase inhibitor is about 350 mg. In some embodiments, the total daily dose of the tyrosine kinase inhibitor is about 360 mg. In some embodiments, the total daily dose of the tyrosine kinase inhibitor is about 370 mg. In some embodiments, the total daily dose of the tyrosine kinase inhibitor is about 375 mg. In some embodiments, the total daily dose of the tyrosine kinase inhibitor is about 380 mg. In some embodiments, the total daily dose of the tyrosine kinase inhibitor is about 400 mg. In some embodiments, the total daily dose of the tyrosine kinase inhibitor is about 410 mg. In some embodiments, the total daily dose of the tyrosine kinase inhibitor is about 420 mg. In some embodiments, the total daily dose of the tyrosine kinase inhibitor is about 425 mg. In some embodiments, the total daily dose of the tyrosine kinase inhibitor is about 430 mg. In some embodiments, the total daily dose of the tyrosine kinase inhibitor is about 440 mg. In some embodiments, the total daily dose of the tyrosine kinase inhibitor is about 450 mg. In some embodiments, the total daily dose of the tyrosine kinase inhibitor is about 460 mg. In some embodiments, the total daily dose of the tyrosine kinase inhibitor is about 470 mg. In some embodiments, the total daily dose of the tyrosine kinase inhibitor is about 475 mg. In some embodiments, the total daily dose of the tyrosine kinase inhibitor is about 480 mg. In some embodiments, the total daily dose of the tyrosine kinase inhibitor is about 490 mg. In some embodiments, the total daily dose of the tyrosine kinase inhibitor is about 500 mg. In some embodiments, the total daily dose of the tyrosine kinase inhibitor is about 525 mg. In some embodiments, the total daily dose of the tyrosine kinase inhibitor is about 550 mg. In some embodiments, the total daily dose of the tyrosine kinase inhibitor is about 575 mg. In some embodiments, the total daily dose of the tyrosine kinase inhibitor is about 600 mg. In some embodiments, the total daily dose of the tyrosine kinase inhibitor is about 625 mg. In some embodiments, the total daily dose of the tyrosine kinase inhibitor is about 650 mg. In some embodiments, the total daily dose of the tyrosine kinase inhibitor is about 675 mg. In some embodiments, the total daily dose of the tyrosine kinase inhibitor is about 700 mg. In some embodiments, the tyrosine kinase inhibitor is rivoceranib. In some embodiments, the tyrosine kinase inhibitor is a pharmaceutically acceptable salt of rivoceranib. In some embodiments, the tyrosine kinase inhibitor is rivoceranib mesylate.

In some embodiments, the total daily dose of rivoceranib is about 10 mg. In some embodiments, the total daily dose of rivoceranib is about 50 mg. In some embodiments, the total daily dose of rivoceranib is about 100 mg. In some embodiments, the total daily dose of rivoceranib is about 150 mg. In some embodiments, the total daily dose of rivoceranib is about 200 mg. In some embodiments, the total daily dose of rivoceranib is about 225 mg. In some embodiments, the total daily dose of rivoceranib is about 250 mg. In some embodiments, the total daily dose of rivoceranib is about 275 mg. In some embodiments, the total daily dose of rivoceranib is about 300 mg. In some embodiments, the total daily dose of rivoceranib is about 310 mg. In some embodiments, the total daily dose of rivoceranib is about 320 mg. In some embodiments, the total daily dose of rivoceranib is about 325 mg. In some embodiments, the total daily dose of rivoceranib is about 330 mg. In some embodiments, the total daily dose of rivoceranib is about 340 mg. In some embodiments, the total daily dose of rivoceranib is about 350 mg. In some embodiments, the total daily dose of rivoceranib is about 360 mg. In some embodiments, the total daily dose of rivoceranib is about 370 mg. In some embodiments, the total daily dose of rivoceranib is about 375 mg. In some embodiments, the total daily dose of rivoceranib is about 380 mg. In some embodiments, the total daily dose of rivoceranib is about 400 mg. In some embodiments, the total daily dose of rivoceranib is about 410 mg. In some embodiments, the total daily dose of rivoceranib is about 420 mg. In some embodiments, the total daily dose of rivoceranib is about 425 mg. In some embodiments, the total daily dose of rivoceranib is about 430 mg. In some embodiments, the total daily dose of rivoceranib is about 440 mg. In some embodiments, the total daily dose of rivoceranib is about 450 mg. In some embodiments, the total daily dose of rivoceranib is about 460 mg. In some embodiments, the total daily dose of rivoceranib is about 470 mg. In some embodiments, the total daily dose of rivoceranib is about 475 mg. In some embodiments, the total daily dose of rivoceranib is about 480 mg. In some embodiments, the total daily dose of rivoceranib is about 490 mg. In some embodiments, the total daily dose of rivoceranib is about 500 mg. In some embodiments, the total daily dose of rivoceranib is about 525 mg. In some embodiments, the total daily dose of rivoceranib is about 550 mg. In some embodiments, the total daily dose of rivoceranib is about 575 mg. In some embodiments, the total daily dose of rivoceranib is about 600 mg. In some embodiments, the total daily dose of rivoceranib is about 625 mg. In some embodiments, the total daily dose of rivoceranib is about 650 mg. In some embodiments, the total daily dose of rivoceranib is about 675 mg. In some embodiments, the total daily dose of rivoceranib is about 700 mg.

In some embodiments, the total daily dose of rivoceranib mesylate is about 10 mg. In some embodiments, the total daily dose of rivoceranib mesylate is about 50 mg. In some embodiments, the total daily dose of rivoceranib mesylate is about 100 mg. In some embodiments, the total daily dose of rivoceranib mesylate is about 150 mg. In some embodiments, the total daily dose of rivoceranib mesylate is about 200 mg. In some embodiments, the total daily dose of rivoceranib mesylate is about 225 mg. In some embodiments, the total daily dose of rivoceranib mesylate is about 250 mg. In some embodiments, the total daily dose of rivoceranib mesylate is about 275 mg. In some embodiments, the total daily dose of rivoceranib mesylate is about 300 mg. In some embodiments, the total daily dose of rivoceranib mesylate is about 310 mg. In some embodiments, the total daily dose of rivoceranib mesylate is about 320 mg. In some embodiments, the total daily dose of rivoceranib mesylate is about 325 mg. In some embodiments, the total daily dose of rivoceranib mesylate is about 330 mg. In some embodiments, the total daily dose of rivoceranib mesylate is about 340 mg. In some embodiments, the total daily dose of rivoceranib mesylate is about 350 mg. In some embodiments, the total daily dose of rivoceranib mesylate is about 360 mg. In some embodiments, the total daily dose of rivoceranib mesylate is about 370 mg. In some embodiments, the total daily dose of rivoceranib mesylate is about 375 mg. In some embodiments, the total daily dose of rivoceranib mesylate is about 380 mg. In some embodiments, the total daily dose of rivoceranib mesylate is about 400 mg. In some embodiments, the total daily dose of rivoceranib mesylate is about 410 mg. In some embodiments, the total daily dose of rivoceranib mesylate is about 420 mg. In some embodiments, the total daily dose of rivoceranib mesylate is about 425 mg. In some embodiments, the total daily dose of rivoceranib mesylate is about 430 mg. In some embodiments, the total daily dose of rivoceranib mesylate is about 440 mg. In some embodiments, the total daily dose of rivoceranib mesylate is about 450 mg. In some embodiments, the total daily dose of rivoceranib mesylate is about 460 mg. In some embodiments, the total daily dose of rivoceranib mesylate is about 470 mg. In some embodiments, the total daily dose of rivoceranib mesylate is about 475 mg. In some embodiments, the total daily dose of rivoceranib mesylate is about 480 mg. In some embodiments, the total daily dose of rivoceranib mesylate is about 490 mg. In some embodiments, the total daily dose of rivoceranib mesylate is about 500 mg. In some embodiments, the total daily dose of rivoceranib mesylate is about 525 mg. In some embodiments, the total daily dose of rivoceranib mesylate is about 550 mg. In some embodiments, the total daily dose of rivoceranib mesylate is about 575 mg. In some embodiments, the total daily dose of rivoceranib mesylate is about 600 mg. In some embodiments, the total daily dose of rivoceranib mesylate is about 625 mg. In some embodiments, the total daily dose of rivoceranib mesylate is about 650 mg. In some embodiments, the total daily dose of rivoceranib mesylate is about 675 mg. In some embodiments, the total daily dose of rivoceranib mesylate is about 700 mg.

In some embodiments, the tyrosine kinase inhibitor is administered orally. In some embodiments, the tyrosine kinase inhibitor is administered in an oral liquid, solid or semisolid dosage form. In some embodiments, the tyrosine kinase inhibitor is administered as a solid oral dosage form. In some embodiments, the tyrosine kinase inhibitor is administered as a pill, tablet, chewable tablet, specialty tablet, buccal tablet, sub-lingual tablet, orally-disintegrating tablet, capsule, gel capsule, soft gel capsule, hard gel capsule, sachet, powder, granule, crystal or orally dispersible film. In some embodiments, the tyrosine kinase inhibitor is administered as a dried powder, a liquid, a capsule, a pellet or a tablet. In some embodiments, the tyrosine kinase inhibitor is administered as a tablet. In some embodiments, the tyrosine kinase inhibitor is administered as a film coated tablet.

In such embodiments, wherein the tyrosine kinase inhibitor is administered as a solid oral dosage form, the tyrosine kinase inhibitor may be admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

In some embodiments, solid dosage forms may be prepared with coatings and shells, such as enteric coatings and others known in the art. They may contain pacifying agents, and can also be of such composition that they release the tyrosine kinase inhibitor in a certain part of the intestinal tract in a delayed manner. Examples of embedded compositions that can be used are polymeric substances and waxes. The tyrosine kinase inhibitor may also be in microencapsulated form, if appropriate, with one or more of the above-mentioned excipients

In some embodiments, the tablet comprises the tyrosine kinase inhibitor in an amount of about 100 mg. In some embodiments, the tablet comprises the tyrosine kinase inhibitor in an amount of about 150 mg. In some embodiments, the tablet comprises the tyrosine kinase inhibitor in an amount of about 200 mg. In some embodiments, the tablet comprises the tyrosine kinase inhibitor in an amount of about 250 mg. In some embodiments, the tablet comprises the tyrosine kinase inhibitor in an amount of about 300 mg. In some embodiments, the tablet comprises the tyrosine kinase inhibitor in an amount of about 350 mg. In some embodiments, the tablet comprises the tyrosine kinase inhibitor in an amount of about 400 mg. In some embodiments, the tablet comprises the tyrosine kinase inhibitor in an amount of about 450 mg. In some embodiments, the tablet comprises the tyrosine kinase inhibitor in an amount of about 500 mg. In some embodiments, the tablet comprises the tyrosine kinase inhibitor in an amount of about 550 mg. In some embodiments, the tablet comprises the tyrosine kinase inhibitor in an amount of about 600 mg. In some embodiments, the tablet comprises the tyrosine kinase inhibitor in an amount of about 650 mg. In some embodiments, the tablet further comprises one or more of pregelatinized starch, microcrystalline cellulose, sodium starch glycolate, povidone (K-30), colloidal silicon dioxide, magnesium stearate and Opadry white.

In some embodiments, the tyrosine kinase inhibitor is administered as a liquid oral dosage form. In some embodiments, the tyrosine kinase inhibitor is administered as a solution, suspension, drink, syrup, elixir, ampoule, dispersion, semi-solid or soft gel.

In some embodiments, the tyrosine kinase inhibitor is administered parenterally. In some embodiments, the tyrosine kinase inhibitor is administered intradermaly, subcutaneously, intramuscularly, intraosseously, intraperitoneally or intravenously. In some embodiments, the tyrosine kinase inhibitor is administered intraperitoneally. In some embodiments, the tyrosine kinase inhibitor is administered intravenously.

In some embodiments, rivoceranib is administered orally. In some embodiments, rivoceranib is administered in an oral liquid, solid or semisolid dosage form. In some embodiments, rivoceranib is administered as a solid oral dosage form. In some embodiments, rivoceranib is administered as a pill, tablet, chewable tablet, specialty tablet, buccal tablet, sub-lingual tablet, orally-disintegrating tablet, capsule, gel capsule, soft gel capsule, hard gel capsule, sachet, powder, granule, crystal or orally dispersible film. In some embodiments, rivoceranib is administered as a dried powder, a liquid, a capsule, a pellet or a tablet. In some embodiments, rivoceranib is administered as a tablet. In some embodiments, rivoceranib is administered as a film coated tablet.

In some embodiments, the tablet comprises rivoceranib in an amount of about 100 mg. In some embodiments, the tablet comprises rivoceranib in an amount of about 150 mg. In some embodiments, the tablet comprises rivoceranib in an amount of about 200 mg. In some embodiments, the tablet comprises rivoceranib in an amount of about 250 mg. In some embodiments, the tablet comprises rivoceranib in an amount of about 300 mg. In some embodiments, the tablet comprises rivoceranib in an amount of about 350 mg. In some embodiments, the tablet comprises rivoceranib in an amount of about 400 mg. In some embodiments, the tablet comprises rivoceranib in an amount of about 450 mg. In some embodiments, the tablet comprises rivoceranib in an amount of about 500 mg. In some embodiments, the tablet comprises rivoceranib in an amount of about 550 mg. In some embodiments, the tablet comprises rivoceranib in an amount of about 600 mg. In some embodiments, the tablet comprises rivoceranib in an amount of about 650 mg. In some embodiments, the tablet further comprises one or more of pregelatinized starch, microcrystalline cellulose, sodium starch glycolate, povidone (K-30), colloidal silicon dioxide, magnesium stearate and Opadry white.

In some embodiments, rivoceranib is administered as a liquid oral dosage form. In some embodiments, rivoceranib is administered as a solution, suspension, drink, syrup, elixir, ampoule, dispersion, semi-solid or soft gel.

In some embodiments, rivoceranib is administered parenterally. In some embodiments, rivoceranib is administered intradermaly, subcutaneously, intramuscularly, intraosseously, intraperitoneally or intravenously. In some embodiments, rivoceranib is administered intraperitoneally. In some embodiments, rivoceranib is administered intravenously.

In some embodiments, rivoceranib mesylate is administered orally. In some embodiments, rivoceranib mesylate is administered in an oral liquid, solid or semisolid dosage form. In some embodiments, rivoceranib mesylate is administered as a solid oral dosage form. In some embodiments, rivoceranib mesylate is administered as a pill, tablet, chewable tablet, specialty tablet, buccal tablet, sub-lingual tablet, orally-disintegrating tablet, capsule, gel capsule, soft gel capsule, hard gel capsule, sachet, powder, granule, crystal or orally dispersible film. In some embodiments, rivoceranib mesylate is administered as a dried powder, a liquid, a capsule, a pellet or a tablet. In some embodiments, rivoceranib mesylate is administered as a tablet. In some embodiments, rivoceranib mesylate is administered as a film coated tablet.

In some embodiments, the tablet comprises rivoceranib mesylate in an amount of about 100 mg. In some embodiments, the tablet comprises rivoceranib mesylate in an amount of about 150 mg. In some embodiments, the tablet comprises rivoceranib mesylate in an amount of about 200 mg. In some embodiments, the tablet comprises rivoceranib mesylate in an amount of about 250 mg. In some embodiments, the tablet comprises rivoceranib mesylate in an amount of about 300 mg. In some embodiments, the tablet comprises rivoceranib mesylate in an amount of about 350 mg. In some embodiments, the tablet comprises rivoceranib mesylate in an amount of about 400 mg. In some embodiments, the tablet comprises rivoceranib mesylate in an amount of about 450 mg. In some embodiments, the tablet comprises rivoceranib mesylate in an amount of about 500 mg. In some embodiments, the tablet comprises rivoceranib mesylate in an amount of about 550 mg. In some embodiments, the tablet comprises rivoceranib mesylate in an amount of about 600 mg. In some embodiments, the tablet comprises rivoceranib mesylate in an amount of about 650 mg. In some embodiments, the tablet further comprises one or more of pregelatinized starch, microcrystalline cellulose, sodium starch glycolate, povidone (K-30), colloidal silicon dioxide, magnesium stearate and Opadry white.

In some embodiments, rivoceranib mesylate is administered as a liquid oral dosage form. In some embodiments, rivoceranib mesylate is administered as a solution, suspension, drink, syrup, elixir, ampoule, dispersion, semi-solid or soft gel.

In some embodiments, rivoceranib mesylate is administered parenterally. In some embodiments, rivoceranib mesylate is administered intradermaly, subcutaneously, intramuscularly, intraosseously, intraperitoneally or intravenously. In some embodiments, rivoceranib mesylate is administered intraperitoneally. In some embodiments, rivoceranib mesylate is administered intravenously.

In some embodiments, the tyrosine kinase inhibitor is administered once daily. In some embodiments, the tyrosine kinase inhibitor is administered twice daily.

In some embodiments, rivoceranib is administered once daily. In some embodiments, rivoceranib is administered twice daily.

In some embodiments, rivoceranib mesylate is administered once daily. In some embodiments, rivoceranib mesylate is administered twice daily.

In one aspect, the disclosure provides a combination therapy comprising apatinib, or a pharmaceutically acceptable salt thereof, and an immunotherapeutic agent. The disclosure also provides a method of treating cancer comprising administering to a patient in need thereof a therapeutically effective amount of apatinib in combination with a therapeutically effective amount of an immunotherapeutic agent. In one aspect, the pharmaceutically acceptable salt is apatinib mesylate.

In one aspect, the immunotherapeutic agent is selected from the group consisting of an antibody, a peptide, pembrolizumab, nivolumab, pidilizumab, BMS-936559, MPDL3280A, MEDI4736, MSB0010718C, atezolizumab, avelumab, durvalumab, ipilimumab, tumor vaccines (e.g., sipuleucel-T), CAR T-cell therapies (e.g., tisagenlecleucel, axicabtagene ciloleucel), and naked monoclonal antibodies (e.g., alemtuzumab).

Cancers treatable by the combination therapies of the disclosure include, but are not limited to lung cancer, small-cell lung cancer, non-small cell lung cancer, carcinoma, lymphoma, blastoma, sarcoma, leukemia, breast cancer, prostate cancer, colon cancer, squamous cell cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

In one aspect, the immunotherapeutic agent is an antibody. In one aspect, the antibody can be a monoclonal or polyclonal antibody. In one aspect, the antibody can be a human or humanized antibody. In one aspect, the antibody can be an anti-programmed death 1 (PD-1) antibody. In one aspect, the antibody can be an anti-muPD-1 antibody. In one aspect, the antibody can be an anti-PD-L1 antibody. In one aspect, the antibody can be an anti-CTLA-4 antibody.

Apatinib, also known as YN968D1, is a tyrosine kinase inhibitor that selectively inhibits the vascular endothelial growth factor receptor-2 (VEGFR2, also known as KDR). It is an orally bioavailable, small molecule agent which is thought to inhibit angiogenesis in cancer cells. In particular, apatinib inhibits VEGF-mediated endothelial cell migration and proliferation thus blocking new blood vessel formation in tumor tissue. This agent also mildly inhibits c-Kit and c-SRC tyrosine kinases. Apatinib mesylate is described in more detail in U.S. Pat. No. 8,362,256, the contents of which are hereby incorporated in their entirety. For the purposes of the disclosure, the term “apatinib” encompasses all pharmaceutically acceptable apatinib salts, and in particular, apatinib mesylate.

### Immunotherapeutic Agents

In some embodiments, described herein are tyrosine kinase inhibitors in combination with an immunotherapeutic agent. In some embodiments, the immunotherapeutic agent is a PD-1 or PD-L1 inhibitor.

The PD-1 (programmed cell death-1) receptor (also known as CD279) is expressed on the surface of activated T cells. Its ligand, PD-L1, is commonly expressed on the surface of dendritic cells or macrophages. In some instances, PD1 and PD-L1 interaction halts or limits the development of the T cell response. When PD-L1 binds to PD-1, an inhibitory signal is transmitted into the T cell, which reduces cytokine production and suppresses T-cell proliferation. In some instances, cancer or tumor cells exploit this signaling pathway as a mechanism to evade detection and inhibit the immune response. In some instances, PD-L1 is overexpressed on cancer or tumor cells or on non-transformed cells in the tumor microenvironment. In some instances, PD-L1 expressed on the tumor cells binds to PD-1 receptors on the activated T cells, which leads to the inhibition of the cytotoxic T cells. These deactivated T cells remain inhibited in the tumor microenvironment. The PD1/PD-L1 pathway represents an adaptive immune resistance mechanism that is exerted by cancer or tumor cells in response to endogenous anti-tumor activity.

PD-1 inhibitors (or anti-PD-1 agents) and PD-L1 inhibitors (or anti-PD-L1 agents) block the interaction between PD-1 and PD-L1 and boost the immune response against cancer cells. In some instances, the blockade of receptor engagement results in the amplification of antigen-specific T cell responses against cancer cells. In some instances, antibodies that block the PD-1/PD-L1 interaction target lymphocyte receptors or their ligands in order to enhance endogenous antitumor activity. In some instances, PD-1 inhibitors and PD-L1 inhibitors overcome distinct immune suppressive pathways within the tumor microenvironment. In some instances, PD-1 inhibitors and/or PD-L1 inhibitors are useful for treating cancer.

Any suitable PD-1 inhibitor or PD-L1 inhibitor may be used in combination with a tyrosine kinase inhibitor described herein. In some embodiments, the PD-1 inhibitor is an antagonist of PD-1. In some embodiments, the PD-L1 inhibitor is an antagonist of PD-L1. In some embodiments, the PD-1 inhibitor or PD-L1 inhibitor is an antibody, variant, or biosimilar thereof. In some embodiments, the PD-1 inhibitor or PD-L1 inhibitor is a monoclonal antibody. In some embodiments, the method of treating cancer with a tyrosine kinase inhibitor described herein in combination with a PD-1 or PD-L1 inhibitor results in a transient reduction in the level of systemic immunosuppression.

Some embodiments provided herein describe a pharmaceutical compositions or methods for use the pharmaceutical compositions comprising a tyrosine kinase inhibitor described herein in combination with a PD-1 inhibitor. PD-1 inhibitors for use in pharmaceutical compositions and methods provided herein include, but are not limited to, nivolumab (Opdivo®), pembrolizumab (Keytruda®), MEDI0680 (AMP-514), AMP-224, AMP-514 (Amplimmune), BGB-A317, PDR001, REGN2810, JS001, AGEN2034, and variants and biosimilars thereof. In some embodiments, the PD-1 inhibitor is to nivolumab (Opdivo®), pembrolizumab (Keytruda®), MEDI0680 (AMP-514), AMP-224, AMP-514 (Amplimmune), or variants or biosimilars thereof. In some embodiments, the PD-1 inhibitor is pidilzumab (CT-011), or a variant or biosimilar thereof. In some embodiments, the PD-1 inhibitor is nivolumab (Opdivo®), or pembrolizumab (Keytruda®), or a variant or biosimilar thereof. In some embodiments, the PD-1 inhibitor is nivolumab (Opdivo®), a nivolumab variant, or a nivolumab biosimilar. In some embodiments, the PD-1 inhibitor is pembrolizumab (Keytruda®), a pembrolizumab variant, or a pembrolizumab biosimilar. In some embodiments, the PD-1 inhibitor is BGB-A317, a BGB-A317 variant, or a BGB-A317 biosimilar. In some embodiments, the PD-1 inhibitor is PDR001, a PDR001 variant, or a PDR001 biosimilar. In some embodiments, the PD-1 inhibitor is REGN2810, a REGN2810 variant, or a REGN2810 biosimilar.

Some embodiments provided herein describe pharmaceutical compositions or methods for using the pharmaceutical compositions comprising a tyrosine kinase inhibitor described herein in combination with a PD-L1 inhibitor. PD-L1 inhibitors for use in pharmaceutical compositions and methods provided herein include but are not limited to Atezolizumab (Tecentriq® or MPDL3280A), avelumab (Bavencio®), Durvalumab (MEDI4736), MPDL3280A (RG7446), BMS-936559 (MDX-1105), MSB0010718C, YW243.55.570, and variants and biosimilars thereof. In some embodiments, the PD-L1 inhibitor is Atezolizumab (Tecentriq® or MPDL3280A), avelumab (Bavencio®), or Durvalumab (MEDI4736), or variants or biosimilars thereof. In some embodiments, the PD-L1 inhibitor is Atezolizumab (Tecentriq® or MPDL3280A) or avelumab (Bavencio®), or a variant or biosimilar thereof. In some embodiments, the PD-L1 inhibitor is atezolizumab (Tecentriq® or MPDL3280A), an atezolizumab variant, or an atezolizumab biosimilar. In some embodiments, the PD-L1 inhibitor is avelumab (Bavencio®), avelumab variant, or an avelumab biosimilar. In some embodiments, the PD-L1 inhibitor is BMS-936559 (MDX-1105), BMS-936559 variant, or a BMS-936559 biosimilar. In some embodiments, the PD-L1 inhibitor is durvalumab (MEDI4736), a durvalumab variant, or a durvalumab biosimilar.

In some embodiments, the combination therapy described herein avoids or reduces adverse or unwanted, serious, or fatal side effects associated with the use of a tyrosine kinase inhibitor and/or a PD-1 or PD-L1 inhibitor. In some embodiments, the combination therapy described herein avoids, reduces, or minimizes (serious) infections, neutropenia, (severe) diarrhea, colon inflammation, colitis, lung tissue inflammation (pneumonitis), intestinal perforation, pneumonia, anemia, thrombocytopenia, nausea, fever, fatigue, cough, abdominal pain, chills, rash, vomiting, hypertriglyceridemia, hyperglycemia, elevated levels of liver enzymes (e.g., ALT and ALST), liver toxicity, swelling in extremities, or a combination thereof in patients receiving the combination therapy. In certain embodiments, the combination therapy described herein avoids, reduces, or minimizes the incidence of infection, including serious infection. In certain embodiments, the combination therapy described herein avoids, reduces, or minimizes the incidence of neutropenia. In certain embodiments, the combination therapy described herein avoids, reduces, or minimizes the incidence of diarrhea, including severe diarrhea. In certain embodiments, the combination therapy described herein avoids, reduces, or minimizes the incidence of colon inflammation. In certain embodiments, the combination therapy described herein avoids, reduces, or minimizes the incidence of colitis. In certain embodiments, the combination therapy described herein avoids, reduces, or minimizes the incidence of lung tissue inflammation (pneumonitis). In certain embodiments, the combination therapy described herein avoids, reduces, or minimizes the incidence of intestinal perforation. In certain embodiments, the combination therapy described herein avoids, reduces, or minimizes the incidence of pneumonia. In certain embodiments, the combination therapy described herein avoids, reduces, or minimizes the incidence of anemia. In certain embodiments, the combination therapy described herein avoids, reduces, or minimizes the incidence of thrombocytopenia. In certain embodiments, the combination therapy described herein avoids, reduces, or minimizes the incidence of nausea. In certain embodiments, the combination therapy described herein avoids, reduces, or minimizes the incidence of fever. In certain embodiments, the combination therapy described herein avoids, reduces, or minimizes the incidence of fatigue. In certain embodiments, the combination therapy described herein avoids, reduces, or minimizes the incidence of cough. In certain embodiments, the combination therapy described herein avoids, reduces, or minimizes the incidence of abdominal pain. In certain embodiments, the combination therapy described herein avoids, reduces, or minimizes the incidence of chills. In certain embodiments, the combination therapy described herein avoids, reduces, or minimizes the incidence of rash. In certain embodiments, the combination therapy described herein avoids, reduces, or minimizes the incidence of vomiting. In certain embodiments, the combination therapy described herein avoids, reduces, or minimizes the incidence of hypertriglyceridemia.

In certain embodiments, the combination therapy described herein avoids, reduces, or minimizes the incidence of hyperglycemia. In certain embodiments, the combination therapy described herein avoids, reduces, or minimizes the incidence of elevated levels of liver enzymes (e.g., ALT and ALST). In certain embodiments, the combination therapy described herein avoids, reduces, or minimizes the incidence of liver toxicity. In certain embodiments, the combination therapy described herein avoids, reduces, or minimizes the incidence of swelling in the extremities.

In some embodiments, the combination therapy described herein avoids or reduces adverse or unwanted side effects associated with chemotherapy, radiotherapy, or cancer therapy. In some instances, the combination therapies and/or compositions described herein provide chemo-protective and/or radio-protective properties to non-cancerous cells. In further or additional embodiments, the lower amount/doses of tyrosine kinase inhibitor reduces or minimizes any undesired side-effects associated with chemotherapy. Non-limiting examples of side-effects associated with chemotherapy, radiotherapy, or cancer therapy include fatigue, anemia, appetite changes, bleeding problems, diarrhea, constipation, hair loss, nausea, vomiting, pain, peripheral neuropathy, swelling, skin and nail changes, urinary and bladder changes, and trouble swallowing.

The present disclosure contemplates the combinational use of apatinib (in particular, apatinib mesylate) with any immunotherapeutic agent that can induce or enhance immune response to cancer.

In particular, the combinational therapy of the disclosure can be used with (but is not limited to) the following immunotherapeutic agents: a peptide, an antibody, pembrolizumab, nivolumab, pidilizumab, BMS-936559, MPDL3280A, MEDI4736, MSB0010718C atezolizumab, avelumab, durvalumab, ipilimumab, tumor vaccines (sipuleucel-T), CAR T-cell therapies (tisagenlecleucel, axicabtagene ciloleucel), and naked monoclonal antibodies (e.g., alemtuzumab).

Some of the preferred antibodies that can be used in the compositions and methods of the present disclosure include anti-muPD-1, anti-PD-L1 and anti-CTLA-4 antibodies.

### Dosing

In some instances drugs dosages are determined as a factor of patient body surface area (BSA). In some instances BSA is a better indicator of metabolic mass than body weight because it is less affected by abnormal adipose mass, e.g., a patient with a larger BSA would presumably have larger organs for a drug to clear through. Indeed, there can be a 4-10 fold variation in drug clearance between individuals.

Various formulae exist, using height and weight, to calculate BSA without direct measurement. The most widely used is the Du Bois formula, which has been shown to be equally as effective in estimating BSA in obese and non-obese patients.

BSA=0.007184×W^{0.425}×H^{0.725}

where W is mass in kg, and H is height in cm.
The average adult male BSA is 2.060 m². The average adult female BSA is 1.830 m². In some instances, pembrolizumab doses are given in units of mg/m². In some instances, nivolumab doses are given in units of mg/m².

In some embodiments, the methods for treating diseases comprise administering a combination of two or more therapies, wherein one of the therapies is an immunotherapeutic agent. In some embodiments, the immunotherapeutic agent is selected from a peptide, an antibody, pembrolizumab, nivolumab, pidilizumab, BMS-936559, MPDL3280A, MEDI4736, MSB0010718C atezolizumab, avelumab, durvalumab, ipilimumab, tumor vaccines (sipuleucel-T), CAR T-cell therapies (tisagenlecleucel, axicabtagene ciloleucel), and naked monoclonal antibodies (e.g., alemtuzumab)

In some embodiments, the immunotherapeutic agent is administered in an amount of about 25 mg/m². In some embodiments, the immunotherapeutic agent is administered in an amount of about 30 mg/m². In some embodiments, the immunotherapeutic agent is administered in an amount of about 35 mg/m². In some embodiments, the immunotherapeutic agent is administered in an amount of about 40 mg/m². In some embodiments, the immunotherapeutic agent is administered in an amount of about 45 mg/m². In some embodiments, the immunotherapeutic agent is administered in an amount of about 50 mg/m². In some embodiments, the immunotherapeutic agent is administered in an amount of about 55 mg/m². In some embodiments, the immunotherapeutic agent is administered in an amount of about 60 mg/m². In some embodiments, the immunotherapeutic agent is administered in an amount of about 65 mg/m². In some embodiments, the immunotherapeutic agent is administered in an amount of about 70 mg/m². In some embodiments, the immunotherapeutic agent is administered in an amount of about 75 mg/m². In some embodiments, the immunotherapeutic agent is administered in an amount of about 80 mg/m². In some embodiments, the immunotherapeutic agent is administered in an amount of about 85 mg/m². In some embodiments, the immunotherapeutic agent is administered in an amount of about 90 mg/m². In some embodiments, the immunotherapeutic agent is administered in an amount of about 95 mg/m². In some embodiments, the immunotherapeutic agent is administered in an amount of about 100 mg/m². In some embodiments, the immunotherapeutic agent is administered in an amount of about 105 mg/m². In some embodiments, the immunotherapeutic agent is administered in an amount of about 110 mg/m². In some embodiments, the immunotherapeutic agent is administered in an amount of about 115 mg/m². In some embodiments, the immunotherapeutic agent is administered in an amount of about 120 mg/m². In some embodiments, the immunotherapeutic agent is administered in an amount of about 125 mg/m². In some embodiments, the immunotherapeutic agent is administered in an amount of about 130 mg/m². In some embodiments, the immunotherapeutic agent is administered in an amount of about 135 mg/m². In some embodiments, the immunotherapeutic agent is administered in an amount of about 140 mg/m². In some embodiments, the immunotherapeutic agent is administered in an amount of about 145 mg/m². In some embodiments, the immunotherapeutic agent is administered in an amount of about 150 mg/m². In some embodiments, the immunotherapeutic agent is administered in an amount of about 160 mg/m². In some embodiments, the immunotherapeutic agent is administered in an amount of about 170 mg/m². In some embodiments, the immunotherapeutic agent is administered in an amount of about 175 mg/m². In some embodiments, the immunotherapeutic agent is administered in an amount of about 180 mg/m². In some embodiments, the immunotherapeutic agent is administered in an amount of about 190 mg/m². In some embodiments, the immunotherapeutic agent is administered in an amount of about 200 mg/m². In some embodiments, the immunotherapeutic agent is a PD-1 or PD-1L inhibitor. In some embodiments, the immunotherapeutic agent is pembrolizumab. In some embodiments, the immunotherapeutic agent is nivolumab.

In some embodiments, the immunotherapeutic agent is administered in an amount of no more than 70 mg/m². In some embodiments, the immunotherapeutic agent is administered in an amount of no more than 80 mg/m². In some embodiments, the immunotherapeutic agent is administered in an amount of no more than 90 mg/m². In some embodiments, the immunotherapeutic agent is administered in an amount of no more than 100 mg/m². In some embodiments, the immunotherapeutic agent is administered in an amount of no more than 125 mg/m². In some embodiments, the immunotherapeutic agent is administered in an amount of no more than 150 mg/m². In some embodiments, the immunotherapeutic agent is administered in an amount of no more than 175 mg/m². In some embodiments, the immunotherapeutic agent is administered in an amount of no more than 200 mg/m². In some embodiments, the immunotherapeutic agent is administered in an amount of no more than 250 mg/m². In some embodiments, the immunotherapeutic agent is a PD-1 or PD-1L inhibitor. In some embodiments, the immunotherapeutic agent is pembrolizumab. In some embodiments, the immunotherapeutic agent is nivolumab.

In some embodiments, pembrolizumab is administered in an amount of about 25 mg/m². In some embodiments, pembrolizumab is administered in an amount of about 30 mg/m². In some embodiments, pembrolizumab is administered in an amount of about 35 mg/m². In some embodiments, pembrolizumab is administered in an amount of about 40 mg/m². In some embodiments, pembrolizumab is administered in an amount of about 45 mg/m². In some embodiments, pembrolizumab is administered in an amount of about 50 mg/m². In some embodiments, pembrolizumab is administered in an amount of about 55 mg/m². In some embodiments, pembrolizumab is administered in an amount of about 60 mg/m². In some embodiments, pembrolizumab is administered in an amount of about 65 mg/m². In some embodiments, pembrolizumab is administered in an amount of about 70 mg/m². In some embodiments, pembrolizumab is administered in an amount of about 75 mg/m². In some embodiments, pembrolizumab is administered in an amount of about 80 mg/m². In some embodiments, pembrolizumab is administered in an amount of about 85 mg/m². In some embodiments, pembrolizumab is administered in an amount of about 90 mg/m². In some embodiments, pembrolizumab is administered in an amount of about 95 mg/m². In some embodiments, pembrolizumab is administered in an amount of about 100 mg/m². In some embodiments, pembrolizumab is administered in an amount of about 105 mg/m². In some embodiments, pembrolizumab is administered in an amount of about 110 mg/m². In some embodiments, pembrolizumab is administered in an amount of about 115 mg/m². In some embodiments, pembrolizumab is administered in an amount of about 120 mg/m². In some embodiments, pembrolizumab is administered in an amount of about 125 mg/m². In some embodiments, pembrolizumab is administered in an amount of about 130 mg/m². In some embodiments, pembrolizumab is administered in an amount of about 135 mg/m². In some embodiments, pembrolizumab is administered in an amount of about 140 mg/m². In some embodiments, pembrolizumab is administered in an amount of about 145 mg/m². In some embodiments, pembrolizumab is administered in an amount of about 150 mg/m². In some embodiments, pembrolizumab is administered in an amount of about 160 mg/m². In some embodiments, pembrolizumab is administered in an amount of about 170 mg/m². In some embodiments, pembrolizumab is administered in an amount of about 175 mg/m². In some embodiments, pembrolizumab is administered in an amount of about 180 mg/m². In some embodiments, pembrolizumab is administered in an amount of about 190 mg/m². In some embodiments, pembrolizumab is administered in an amount of about 200 mg/m². In some embodiments, pembrolizumab is administered in an amount of about 210 mg/m². In some embodiments, pembrolizumab is administered in an amount of about 220 mg/m². In some embodiments, pembrolizumab is administered in an amount of about 230 mg/m². In some embodiments, pembrolizumab is administered in an amount of about 240 mg/m². In some embodiments, pembrolizumab is administered in an amount of about 250 mg/m². In some embodiments, pembrolizumab is administered in an amount of about 260 mg/m². In some embodiments, pembrolizumab is administered in an amount of about 270 mg/m². In some embodiments, pembrolizumab is administered in an amount of about 280 mg/m². In some embodiments, pembrolizumab is administered in an amount of about 290 mg/m². In some embodiments, pembrolizumab is administered in an amount of about 300 mg/m². In some embodiments, pembrolizumab is administered in an amount of about 310 mg/m². In some embodiments, pembrolizumab is administered in an amount of about 320 mg/m². In some embodiments, pembrolizumab is administered in an amount of about 330 mg/m². In some embodiments, pembrolizumab is administered in an amount of about 340 mg/m². In some embodiments, pembrolizumab is administered in an amount of about 350 mg/m². In some embodiments, pembrolizumab is administered in an amount of about 375 mg/m². In some embodiments, pembrolizumab is administered in an amount of about 400 mg/m². In some embodiments, pembrolizumab is administered in an amount of about 425 mg/m². In some embodiments, pembrolizumab is administered in an amount of about 450 mg/m². In some embodiments, pembrolizumab is administered in an amount of about 475 mg/m². In some embodiments, pembrolizumab is administered in an amount of about 500 mg/m². In some embodiments, pembrolizumab is administered in an amount of about 525 mg/m². In some embodiments, pembrolizumab is administered in an amount of about 550 mg/m². In some embodiments, pembrolizumab is administered in an amount of about 575 mg/m². In some embodiments, pembrolizumab is administered in an amount of about 600 mg/m².

In some embodiments, pembrolizumab is administered in an amount of no more than 70 mg/m². In some embodiments, pembrolizumab is administered in an amount of no more than 80 mg/m². In some embodiments, pembrolizumab is administered in an amount of no more than 90 mg/m². In some embodiments, pembrolizumab is administered in an amount of no more than 100 mg/m². In some embodiments, pembrolizumab is administered in an amount of no more than 125 mg/m². In some embodiments, pembrolizumab is administered in an amount of no more than 150 mg/m². In some embodiments, pembrolizumab is administered in an amount of no more than 175 mg/m². In some embodiments, pembrolizumab is administered in an amount of no more than 200 mg/m². In some embodiments, pembrolizumab is administered in an amount of no more than 250 mg/m². In some embodiments, pembrolizumab is administered in an amount of no more than 300 mg/m². In some embodiments, pembrolizumab is administered in an amount of no more than 350 mg/m². In some embodiments, pembrolizumab is administered in an amount of no more than 400 mg/m².

In some embodiments, nivolumab is administered in an amount of about 25 mg/m². In some embodiments, nivolumab is administered in an amount of about 30 mg/m². In some embodiments, nivolumab is administered in an amount of about 35 mg/m². In some embodiments, nivolumab is administered in an amount of about 40 mg/m². In some embodiments, nivolumab is administered in an amount of about 45 mg/m². In some embodiments, nivolumab is administered in an amount of about 50 mg/m². In some embodiments, nivolumab is administered in an amount of about 55 mg/m². In some embodiments, nivolumab is administered in an amount of about 60 mg/m². In some embodiments, nivolumab is administered in an amount of about 65 mg/m². In some embodiments, nivolumab is administered in an amount of about 70 mg/m². In some embodiments, nivolumab is administered in an amount of about 75 mg/m². In some embodiments, nivolumab is administered in an amount of about 80 mg/m². In some embodiments, nivolumab is administered in an amount of about 85 mg/m². In some embodiments, nivolumab is administered in an amount of about 90 mg/m². In some embodiments, nivolumab is administered in an amount of about 95 mg/m². In some embodiments, nivolumab is administered in an amount of about 100 mg/m². In some embodiments, nivolumab is administered in an amount of about 105 mg/m². In some embodiments, nivolumab is administered in an amount of about 110 mg/m². In some embodiments, nivolumab is administered in an amount of about 115 mg/m². In some embodiments, nivolumab is administered in an amount of about 120 mg/m². In some embodiments, nivolumab is administered in an amount of about 125 mg/m². In some embodiments, nivolumab is administered in an amount of about 130 mg/m². In some embodiments, nivolumab is administered in an amount of about 135 mg/m². In some embodiments, nivolumab is administered in an amount of about 140 mg/m². In some embodiments, nivolumab is administered in an amount of about 145 mg/m². In some embodiments, nivolumab is administered in an amount of about 150 mg/m². In some embodiments, nivolumab is administered in an amount of about 160 mg/m². In some embodiments, nivolumab is administered in an amount of about 170 mg/m². In some embodiments, nivolumab is administered in an amount of about 175 mg/m². In some embodiments, nivolumab is administered in an amount of about 180 mg/m². In some embodiments, nivolumab is administered in an amount of about 190 mg/m². In some embodiments, nivolumab is administered in an amount of about 200 mg/m². In some embodiments, nivolumab is administered in an amount of about 210 mg/m². In some embodiments, nivolumab is administered in an amount of about 220 mg/m². In some embodiments, nivolumab is administered in an amount of about 230 mg/m². In some embodiments, nivolumab is administered in an amount of about 240 mg/m². In some embodiments, nivolumab is administered in an amount of about 250 mg/m². In some embodiments, nivolumab is administered in an amount of about 260 mg/m². In some embodiments, nivolumab is administered in an amount of about 270 mg/m². In some embodiments, nivolumab is administered in an amount of about 280 mg/m². In some embodiments, nivolumab is administered in an amount of about 290 mg/m². In some embodiments, nivolumab is administered in an amount of about 300 mg/m². In some embodiments, nivolumab is administered in an amount of about 310 mg/m². In some embodiments, nivolumab is administered in an amount of about 320 mg/m². In some embodiments, nivolumab is administered in an amount of about 330 mg/m². In some embodiments, nivolumab is administered in an amount of about 340 mg/m². In some embodiments, nivolumab is administered in an amount of about 350 mg/m². In some embodiments, nivolumab is administered in an amount of about 375 mg/m². In some embodiments, nivolumab is administered in an amount of about 400 mg/m². In some embodiments, nivolumab is administered in an amount of about 425 mg/m². In some embodiments, nivolumab is administered in an amount of about 450 mg/m². In some embodiments, nivolumab is administered in an amount of about 475 mg/m². In some embodiments, nivolumab is administered in an amount of about 500 mg/m². In some embodiments, nivolumab is administered in an amount of about 525 mg/m². In some embodiments, nivolumab is administered in an amount of about 550 mg/m². In some embodiments, nivolumab is administered in an amount of about 575 mg/m². In some embodiments, nivolumab is administered in an amount of about 600 mg/m².

In some embodiments, nivolumab is administered in an amount of no more than 70 mg/m². In some embodiments, nivolumab is administered in an amount of no more than 80 mg/m². In some embodiments, nivolumab is administered in an amount of no more than 90 mg/m². In some embodiments, nivolumab is administered in an amount of no more than 100 mg/m². In some embodiments, nivolumab is administered in an amount of no more than 125 mg/m². In some embodiments, nivolumab is administered in an amount of no more than 150 mg/m². In some embodiments, nivolumab is administered in an amount of no more than 175 mg/m². In some embodiments, nivolumab is administered in an amount of no more than 200 mg/m². In some embodiments, nivolumab is administered in an amount of no more than 250 mg/m². In some embodiments, nivolumab is administered in an amount of no more than 300 mg/m². In some embodiments, nivolumab is administered in an amount of no more than 350 mg/m². In some embodiments, nivolumab is administered in an amount of no more than 400 mg/m².

In some embodiments, the immunotherapeutic agent is administered in a dosage amount of about 25 mg. In some embodiments, the immunotherapeutic agent is administered in a dosage amount of about 30 mg. In some embodiments, the immunotherapeutic agent is administered in a dosage amount of about 35 mg. In some embodiments, the immunotherapeutic agent is administered in a dosage amount of about 40 mg. In some embodiments, the immunotherapeutic agent is administered in a dosage amount of about 45 mg. In some embodiments, the immunotherapeutic agent is administered in a dosage amount of about 50 mg. In some embodiments, the immunotherapeutic agent is administered in a dosage amount of about 55 mg. In some embodiments, the immunotherapeutic agent is administered in a dosage amount of about 60 mg. In some embodiments, the immunotherapeutic agent is administered in a dosage amount of about 65 mg. In some embodiments, the immunotherapeutic agent is administered in a dosage amount of about 70 mg. In some embodiments, the immunotherapeutic agent is administered in a dosage amount of about 75 mg. In some embodiments, the immunotherapeutic agent is administered in a dosage amount of about 80 mg. In some embodiments, the immunotherapeutic agent is administered in a dosage amount of about 85 mg. In some embodiments, the immunotherapeutic agent is administered in a dosage amount of about 90 mg. In some embodiments, the immunotherapeutic agent is administered in a dosage amount of about 95 mg. In some embodiments, the immunotherapeutic agent is administered in a dosage amount of about 100 mg. In some embodiments, the immunotherapeutic agent is administered in a dosage amount of about 105 mg. In some embodiments, the immunotherapeutic agent is administered in a dosage amount of about 110 mg. In some embodiments, the immunotherapeutic agent is administered in a dosage amount of about 115 mg. In some embodiments, the immunotherapeutic agent is administered in a dosage amount of about 120 mg. In some embodiments, the immunotherapeutic agent is administered in a dosage amount of about 125 mg. In some embodiments, the immunotherapeutic agent is administered in a dosage amount of about 130 mg. In some embodiments, the immunotherapeutic agent is administered in a dosage amount of about 135 mg. In some embodiments, the immunotherapeutic agent is administered in a dosage amount of about 140 mg. In some embodiments, the immunotherapeutic agent is administered in a dosage amount of about 145 mg. In some embodiments, the immunotherapeutic agent is administered in a dosage amount of about 150 mg. In some embodiments, the immunotherapeutic agent is administered in a dosage amount of about 160 mg. In some embodiments, the immunotherapeutic agent is administered in a dosage amount of about 170 mg. In some embodiments, the immunotherapeutic agent is administered in a dosage amount of about 175 mg. In some embodiments, the immunotherapeutic agent is administered in a dosage amount of about 180 mg. In some embodiments, the immunotherapeutic agent is administered in a dosage amount of about 190 mg. In some embodiments, the immunotherapeutic agent is administered in a dosage amount of about 200 mg. In some embodiments, the immunotherapeutic agent is a PD-1 or PD-1L inhibitor. In some embodiments, the immunotherapeutic agent is pembrolizumab. In some embodiments, the immunotherapeutic agent is nivolumab.

In some embodiments, the immunotherapeutic agent is administered in a dosage amount of no more than 70 mg. In some embodiments, the immunotherapeutic agent is administered in a dosage amount of no more than 80 mg. In some embodiments, the immunotherapeutic agent is administered in a dosage amount of no more than 90 mg. In some embodiments, the immunotherapeutic agent is administered in a dosage amount of no more than 100 mg. In some embodiments, the immunotherapeutic agent is administered in a dosage amount of no more than 125 mg. In some embodiments, the immunotherapeutic agent is administered in a dosage amount of no more than 150 mg. In some embodiments, the immunotherapeutic agent is administered in a dosage amount of no more than 175 mg. In some embodiments, the immunotherapeutic agent is administered in a dosage amount of no more than 200 mg. In some embodiments, the immunotherapeutic agent is administered in a dosage amount of no more than 250 mg. In some embodiments, the immunotherapeutic agent is a PD-1 or PD-1L inhibitor. In some embodiments, the immunotherapeutic agent is pembrolizumab. In some embodiments, the immunotherapeutic agent is nivolumab.

In some embodiments, pembrolizumab is administered in a dosage amount of about 25 mg. In some embodiments, pembrolizumab is administered in a dosage amount of about 30 mg. In some embodiments, pembrolizumab is administered in a dosage amount of about 35 mg. In some embodiments, pembrolizumab is administered in a dosage amount of about 40 mg. In some embodiments, pembrolizumab is administered in a dosage amount of about 45 mg. In some embodiments, pembrolizumab is administered in a dosage amount of about 50 mg. In some embodiments, pembrolizumab is administered in a dosage amount of about 55 mg. In some embodiments, pembrolizumab is administered in a dosage amount of about 60 mg. In some embodiments, pembrolizumab is administered in a dosage amount of about 65 mg. In some embodiments, pembrolizumab is administered in a dosage amount of about 70 mg. In some embodiments, pembrolizumab is administered in a dosage amount of about 75 mg. In some embodiments, pembrolizumab is administered in a dosage amount of about 80 mg. In some embodiments, pembrolizumab is administered in a dosage amount of about 85 mg. In some embodiments, pembrolizumab is administered in a dosage amount of about 90 mg. In some embodiments, pembrolizumab is administered in a dosage amount of about 95 mg. In some embodiments, pembrolizumab is administered in a dosage amount of about 100 mg. In some embodiments, pembrolizumab is administered in a dosage amount of about 105 mg. In some embodiments, pembrolizumab is administered in a dosage amount of about 110 mg. In some embodiments, pembrolizumab is administered in a dosage amount of about 115 mg. In some embodiments, pembrolizumab is administered in a dosage amount of about 120 mg. In some embodiments, pembrolizumab is administered in a dosage amount of about 125 mg. In some embodiments, pembrolizumab is administered in a dosage amount of about 130 mg. In some embodiments, pembrolizumab is administered in a dosage amount of about 135 mg. In some embodiments, pembrolizumab is administered in a dosage amount of about 140 mg. In some embodiments, pembrolizumab is administered in a dosage amount of about 145 mg. In some embodiments, pembrolizumab is administered in a dosage amount of about 150 mg. In some embodiments, pembrolizumab is administered in a dosage amount of about 160 mg. In some embodiments, pembrolizumab is administered in a dosage amount of about 170 mg. In some embodiments, pembrolizumab is administered in a dosage amount of about 175 mg. In some embodiments, pembrolizumab is administered in a dosage amount of about 180 mg. In some embodiments, pembrolizumab is administered in a dosage amount of about 190 mg. In some embodiments, pembrolizumab is administered in a dosage amount of about 200 mg. In some embodiments, pembrolizumab is administered in a dosage amount of about 210 mg. In some embodiments, pembrolizumab is administered in a dosage amount of about 220 mg. In some embodiments, pembrolizumab is administered in a dosage amount of about 230 mg. In some embodiments, pembrolizumab is administered in a dosage amount of about 240 mg. In some embodiments, pembrolizumab is administered in a dosage amount of about 250 mg. In some embodiments, pembrolizumab is administered in a dosage amount of about 260 mg. In some embodiments, pembrolizumab is administered in a dosage amount of about 270 mg. In some embodiments, pembrolizumab is administered in a dosage amount of about 280 mg. In some embodiments, pembrolizumab is administered in a dosage amount of about 290 mg. In some embodiments, pembrolizumab is administered in a dosage amount of about 300 mg. In some embodiments, pembrolizumab is administered in a dosage amount of about 310 mg. In some embodiments, pembrolizumab is administered in a dosage amount of about 320 mg. In some embodiments, pembrolizumab is administered in a dosage amount of about 330 mg. In some embodiments, pembrolizumab is administered in a dosage amount of about 340 mg. In some embodiments, pembrolizumab is administered in a dosage amount of about 350 mg. In some embodiments, pembrolizumab is administered in a dosage amount of about 375 mg. In some embodiments, pembrolizumab is administered in a dosage amount of about 400 mg. In some embodiments, pembrolizumab is administered in a dosage amount of about 425 mg. In some embodiments, pembrolizumab is administered in a dosage amount of about 450 mg. In some embodiments, pembrolizumab is administered in a dosage amount of about 475 mg. In some embodiments, pembrolizumab is administered in a dosage amount of about 500 mg. In some embodiments, pembrolizumab is administered in a dosage amount of about 525 mg. In some embodiments, pembrolizumab is administered in a dosage amount of about 550 mg. In some embodiments, pembrolizumab is administered in a dosage amount of about 575 mg. In some embodiments, pembrolizumab is administered in a dosage amount of about 600 mg.

In some embodiments, pembrolizumab is administered in a dosage amount of no more than 70 mg. In some embodiments, pembrolizumab is administered in a dosage amount of no more than 80 mg. In some embodiments, pembrolizumab is administered in a dosage amount of no more than 90 mg. In some embodiments, pembrolizumab is administered in a dosage amount of no more than 100 mg. In some embodiments, pembrolizumab is administered in a dosage amount of no more than 125 mg. In some embodiments, pembrolizumab is administered in a dosage amount of no more than 150 mg. In some embodiments, pembrolizumab is administered in a dosage amount of no more than 175 mg. In some embodiments, pembrolizumab is administered in a dosage amount of no more than 200 mg. In some embodiments, pembrolizumab is administered in a dosage amount of no more than 250 mg. In some embodiments, pembrolizumab is administered in a dosage amount of no more than 300 mg. In some embodiments, pembrolizumab is administered in a dosage amount of no more than 350 mg. In some embodiments, pembrolizumab is administered in a dosage amount of no more than 400 mg. In some embodiments, pembrolizumab is administered in a dosage amount of no more than 425 mg. In some embodiments, pembrolizumab is administered in a dosage amount of no more than 450 mg. In some embodiments, pembrolizumab is administered in a dosage amount of no more than 475 mg. In some embodiments, pembrolizumab is administered in a dosage amount of no more than 500 mg.

In some embodiments, nivolumab is administered in a dosage amount of about 25 mg. In some embodiments, nivolumab is administered in a dosage amount of about 30 mg. In some embodiments, nivolumab is administered in a dosage amount of about 35 mg. In some embodiments, nivolumab is administered in a dosage amount of about 40 mg. In some embodiments, nivolumab is administered in a dosage amount of about 45 mg. In some embodiments, nivolumab is administered in a dosage amount of about 50 mg. In some embodiments, nivolumab is administered in a dosage amount of about 55 mg. In some embodiments, nivolumab is administered in a dosage amount of about 60 mg. In some embodiments, nivolumab is administered in a dosage amount of about 65 mg. In some embodiments, nivolumab is administered in a dosage amount of about 70 mg. In some embodiments, nivolumab is administered in a dosage amount of about 75 mg. In some embodiments, nivolumab is administered in a dosage amount of about 80 mg. In some embodiments, nivolumab is administered in a dosage amount of about 85 mg. In some embodiments, nivolumab is administered in a dosage amount of about 90 mg. In some embodiments, nivolumab is administered in a dosage amount of about 95 mg. In some embodiments, nivolumab is administered in a dosage amount of about 100 mg. In some embodiments, nivolumab is administered in a dosage amount of about 105 mg. In some embodiments, nivolumab is administered in a dosage amount of about 110 mg. In some embodiments, nivolumab is administered in a dosage amount of about 115 mg. In some embodiments, nivolumab is administered in a dosage amount of about 120 mg. In some embodiments, nivolumab is administered in a dosage amount of about 125 mg. In some embodiments, nivolumab is administered in a dosage amount of about 130 mg. In some embodiments, nivolumab is administered in a dosage amount of about 135 mg. In some embodiments, nivolumab is administered in a dosage amount of about 140 mg. In some embodiments, nivolumab is administered in a dosage amount of about 145 mg. In some embodiments, nivolumab is administered in a dosage amount of about 150 mg. In some embodiments, nivolumab is administered in a dosage amount of about 160 mg. In some embodiments, nivolumab is administered in a dosage amount of about 170 mg. In some embodiments, nivolumab is administered in a dosage amount of about 175 mg. In some embodiments, nivolumab is administered in a dosage amount of about 180 mg. In some embodiments, nivolumab is administered in a dosage amount of about 190 mg. In some embodiments, nivolumab is administered in a dosage amount of about 200 mg. In some embodiments, nivolumab is administered in a dosage amount of about 210 mg. In some embodiments, nivolumab is administered in a dosage amount of about 220 mg. In some embodiments, nivolumab is administered in a dosage amount of about 230 mg. In some embodiments, nivolumab is administered in a dosage amount of about 240 mg. In some embodiments, nivolumab is administered in a dosage amount of about 250 mg. In some embodiments, nivolumab is administered in a dosage amount of about 260 mg. In some embodiments, nivolumab is administered in a dosage amount of about 270 mg. In some embodiments, nivolumab is administered in a dosage amount of about 280 mg. In some embodiments, nivolumab is administered in a dosage amount of about 290 mg. In some embodiments, nivolumab is administered in a dosage amount of about 300 mg. In some embodiments, nivolumab is administered in a dosage amount of about 310 mg. In some embodiments, nivolumab is administered in a dosage amount of about 320 mg. In some embodiments, nivolumab is administered in a dosage amount of about 330 mg. In some embodiments, nivolumab is administered in a dosage amount of about 340 mg. In some embodiments, nivolumab is administered in a dosage amount of about 350 mg. In some embodiments, nivolumab is administered in a dosage amount of about 375 mg. In some embodiments, nivolumab is administered in a dosage amount of about 400 mg. In some embodiments, nivolumab is administered in a dosage amount of about 425 mg. In some embodiments, nivolumab is administered in a dosage amount of about 450 mg. In some embodiments, nivolumab is administered in a dosage amount of about 475 mg. In some embodiments, nivolumab is administered in a dosage amount of about 500 mg. In some embodiments, nivolumab is administered in a dosage amount of about 525 mg. In some embodiments, nivolumab is administered in a dosage amount of about 550 mg. In some embodiments, nivolumab is administered in a dosage amount of about 575 mg. In some embodiments, nivolumab is administered in a dosage amount of about 600 mg.

In some embodiments, nivolumab is administered in a dosage amount of no more than 70 mg. In some embodiments, nivolumab is administered in a dosage amount of no more than 80 mg. In some embodiments, nivolumab is administered in a dosage amount of no more than 90 mg. In some embodiments, nivolumab is administered in a dosage amount of no more than 100 mg. In some embodiments, nivolumab is administered in a dosage amount of no more than 125 mg. In some embodiments, nivolumab is administered in a dosage amount of no more than 150 mg. In some embodiments, nivolumab is administered in a dosage amount of no more than 175 mg. In some embodiments, nivolumab is administered in a dosage amount of no more than 200 mg. In some embodiments, nivolumab is administered in a dosage amount of no more than 250 mg. In some embodiments, nivolumab is administered in a dosage amount of no more than 300 mg. In some embodiments, nivolumab is administered in a dosage amount of no more than 350 mg. In some embodiments, nivolumab is administered in a dosage amount of no more than 400 mg. In some embodiments, nivolumab is administered in a dosage amount of no more than 425 mg. In some embodiments, nivolumab is administered in a dosage amount of no more than 450 mg. In some embodiments, nivolumab is administered in a dosage amount of no more than 475 mg. In some embodiments, nivolumab is administered in a dosage amount of no more than 500 mg.

In some embodiments, the immunotherapeutic agent is administered in an amount of from 10 mg/m² to 150 mg/m². In some embodiments, the immunotherapeutic agent is administered in an amount of from 20 mg/m² to 140 mg/m². In some embodiments, the immunotherapeutic agent is administered in an amount of from 30 mg/m² to 130 mg/m². In some embodiments, the immunotherapeutic agent is administered in an amount of from 40 mg/m² to 125 mg/m². In some embodiments, the immunotherapeutic agent is administered in an amount of from 50 mg/m² to 110 mg/m². In some embodiments, the immunotherapeutic agent is administered in an amount of from 50 mg/m² to 90 mg/m². In some embodiments, the immunotherapeutic agent is administered in an amount of from 60 mg/m² to 100 mg/m². In some embodiments, the immunotherapeutic agent is administered in an amount of from 65 mg/m² to 95 mg/m². In some embodiments, the immunotherapeutic agent is administered in an amount of from 70 mg/m² to 90 mg/m². In some embodiments, the immunotherapeutic agent is a PD-1 or PD-1L inhibitor. In some embodiments, the immunotherapeutic agent is pembrolizumab. In some embodiments, the immunotherapeutic agent is nivolumab.

In some embodiments, pembrolizumab is administered in an amount of from 10 mg/m² to 150 mg/m². In some embodiments, pembrolizumab is administered in an amount of from 20 mg/m² to 140 mg/m². In some embodiments, pembrolizumab is administered in an amount of from 30 mg/m² to 130 mg/m². In some embodiments, pembrolizumab is administered in an amount of from 40 mg/m² to 125 mg/m². In some embodiments, pembrolizumab is administered in an amount of from 50 mg/m² to 110 mg/m². In some embodiments, pembrolizumab is administered in an amount of from 50 mg/m² to 90 mg/m². In some embodiments, pembrolizumab is administered in an amount of from 60 mg/m² to 100 mg/m². In some embodiments, pembrolizumab is administered in an amount of from 65 mg/m² to 95 mg/m². In some embodiments, pembrolizumab is administered in an amount of from 70 mg/m² to 90 mg/m².

In some embodiments, nivolumab is administered in an amount of from 10 mg/m² to 150 mg/m². In some embodiments, nivolumab is administered in an amount of from 20 mg/m² to 140 mg/m². In some embodiments, nivolumab is administered in an amount of from 30 mg/m² to 130 mg/m². In some embodiments, nivolumab is administered in an amount of from 40 mg/m² to 125 mg/m². In some embodiments, nivolumab is administered in an amount of from 50 mg/m² to 110 mg/m². In some embodiments, nivolumab is administered in an amount of from 50 mg/m² to 90 mg/m². In some embodiments, nivolumab is administered in an amount of from 60 mg/m² to 100 mg/m². In some embodiments, nivolumab is administered in an amount of from 65 mg/m² to 95 mg/m². In some embodiments, nivolumab is administered in an amount of from 70 mg/m² to 90 mg/m².

In some embodiments, pembrolizumab is administered in a dosage amount of from 10 mg to 100 mg. In some embodiments, pembrolizumab is administered in a dosage amount of from 50 mg to 150 mg. In some embodiments, pembrolizumab is administered in a dosage amount of from 75 mg to 175 mg. In some embodiments, pembrolizumab is administered in a dosage amount of from 100 mg to 200 mg. In some embodiments, pembrolizumab is administered in a dosage amount of from 125 mg to 225 mg. In some embodiments, pembrolizumab is administered in a dosage amount of from 150 mg to 250 mg. In some embodiments, pembrolizumab is administered in a dosage amount of from 175 mg to 275 mg. In some embodiments, pembrolizumab is administered in a dosage amount of from 200 mg to 300 mg. In some embodiments, pembrolizumab is administered in a dosage amount of from 225 mg to 325 mg. In some embodiments, pembrolizumab is administered in a dosage amount of from 250 mg to 350 mg. In some embodiments, pembrolizumab is administered in a dosage amount of from 275 mg to 375 mg. In some embodiments, pembrolizumab is administered in a dosage amount of from 300 mg to 400 mg. In some embodiments, pembrolizumab is administered in a dosage amount of from 100 mg to 300 mg. In some embodiments, pembrolizumab is administered in a dosage amount of from 200 mg to 400 mg.

In some embodiments, nivolumab is administered in a dosage amount of from 10 mg to 100 mg. In some embodiments, nivolumab is administered in a dosage amount of from 50 mg to 150 mg. In some embodiments, nivolumab is administered in a dosage amount of from 75 mg to 175 mg. In some embodiments, nivolumab is administered in a dosage amount of from 100 mg to 200 mg. In some embodiments, nivolumab is administered in a dosage amount of from 125 mg to 225 mg. In some embodiments, nivolumab is administered in a dosage amount of from 150 mg to 250 mg. In some embodiments, nivolumab is administered in a dosage amount of from 175 mg to 275 mg. In some embodiments, nivolumab is administered in a dosage amount of from 200 mg to 300 mg. In some embodiments, nivolumab is administered in a dosage amount of from 225 mg to 325 mg. In some embodiments, nivolumab is administered in a dosage amount of from 250 mg to 350 mg. In some embodiments, nivolumab is administered in a dosage amount of from 275 mg to 375 mg. In some embodiments, nivolumab is administered in a dosage amount of from 300 mg to 400 mg. In some embodiments, nivolumab is administered in a dosage amount of from 100 mg to 300 mg. In some embodiments, nivolumab is administered in a dosage amount of from 200 mg to 400 mg.

In some embodiments, the immunotherapeutic agent is administered orally or parenterally. In some embodiments, the immunotherapeutic agent is administered orally. In some embodiments, the immunotherapeutic agent is administered parenterally. In some embodiments, the immunotherapeutic agent is administered intravenously. In some embodiments, the immunotherapeutic agent is a PD-1 or PD-1L inhibitor. In some embodiments, the immunotherapeutic agent is pembrolizumab. In some embodiments, the immunotherapeutic agent is nivolumab.

In some embodiments, pembrolizumab is administered orally or parenterally. In some embodiments, pembrolizumab is administered orally. In some embodiments, pembrolizumab is administered parenterally. In some embodiments, pembrolizumab is administered intravenously.

In some embodiments, nivolumab is administered orally or parenterally. In some embodiments, nivolumab is administered orally. In some embodiments, nivolumab is administered parenterally. In some embodiments, nivolumab is administered intravenously.

In some embodiments, the immunotherapeutic agent is administered over a period of less than one hour. In some embodiments, the immunotherapeutic agent is administered over a period of about one hour. In some embodiments, the immunotherapeutic agent is administered over a period of about 1.5 hours. In some embodiments, the immunotherapeutic agent is administered over a period of about two hours. In some embodiments, the immunotherapeutic agent is administered over a period of about the hours. In some embodiments, the immunotherapeutic agent is administered over a period of less than two hours. In some embodiments, the immunotherapeutic agent is administered over a period of 30-60 minutes. In some embodiments, the immunotherapeutic agent is administered over a period of about 45 minutes. In some embodiments, the immunotherapeutic agent is a PD-1 or PD-1L inhibitor. In some embodiments, the immunotherapeutic agent is pembrolizumab. In some embodiments, the immunotherapeutic agent is nivolumab.

In some embodiments, pembrolizumab is administered over a period of less than one hour. In some embodiments, pembrolizumab is administered over a period of about one hour. In some embodiments, pembrolizumab is administered over a period of about 1.5 hour. In some embodiments, pembrolizumab is administered over a period of about two hours. In some embodiments, pembrolizumab administered over a period of about the hours. In some embodiments, pembrolizumab is administered over a period of less than two hours. In some embodiments, pembrolizumab is administered over a period of 30-60 minutes. In some embodiments, pembrolizumab is administered over a period of about 45 minutes.

In some embodiments, pembrolizumab mesylate is administered over a period of less than one hour. In some embodiments, pembrolizumab mesylate is administered over a period of about one hour. In some embodiments, pembrolizumab mesylate is administered over a period of about 1.5 hour. In some embodiments, pembrolizumab mesylate is administered over a period of about two hours. In some embodiments, pembrolizumab mesylate administered over a period of about the hours. In some embodiments, pembrolizumab mesylate is administered over a period of less than two hours. In some embodiments, pembrolizumab mesylate is administered over a period of 30-60 minutes. In some embodiments, pembrolizumab mesylate is administered over a period of about 45 minutes.

In some embodiments, nivolumab is administered over a period of less than one hour. In some embodiments, nivolumab is administered over a period of about one hour. In some embodiments, nivolumab is administered over a period of about 1.5 hour. In some embodiments, nivolumab is administered over a period of about two hours. In some embodiments, nivolumab administered over a period of about the hours. In some embodiments, nivolumab is administered over a period of less than two hours. In some embodiments, nivolumab is administered over a period of 30-60 minutes. In some embodiments, nivolumab is administered over a period of about 45 minutes.

In some embodiments, nivolumab mesylate is administered over a period of less than one hour. In some embodiments, nivolumab mesylate is administered over a period of about one hour. In some embodiments, nivolumab mesylate is administered over a period of about 1.5 hour. In some embodiments, nivolumab mesylate is administered over a period of about two hours. In some embodiments, nivolumab mesylate administered over a period of about the hours. In some embodiments, nivolumab mesylate is administered over a period of less than two hours. In some embodiments, nivolumab mesylate is administered over a period of 30-60 minutes. In some embodiments, nivolumab mesylate is administered over a period of about 45 minutes.

In some embodiments, the immunotherapeutic agent is administered no more than once a week. In some embodiments, the immunotherapeutic agent is administered at least once a week. In some embodiments, the immunotherapeutic agent is administered once a week. In some embodiments, the immunotherapeutic agent is administered twice a month. In some embodiments, the immunotherapeutic agent is administered three times a month. In some embodiments, the immunotherapeutic agent is administered once a month. In some embodiments, the immunotherapeutic agent is administered in a 28 day cycle, once a week for 3 weeks. In some embodiments, the immunotherapeutic agent is a PD-1 or PD-1L inhibitor. In some embodiments, the immunotherapeutic agent is pembrolizumab. In some embodiments, the immunotherapeutic agent is nivolumab.

In some embodiments, pembrolizumab is administered no more than once a week. In some embodiments, pembrolizumab is administered at least once a week. In some embodiments, pembrolizumab is administered once a week. In some embodiments, pembrolizumab is administered twice a month. In some embodiments, pembrolizumab is administered three times a month. In some embodiments, pembrolizumab is administered once a month. In some embodiments, pembrolizumab is administered in a 28 day cycle, once a week for 3 weeks.

In some embodiments, nivolumab is administered no more than once a week. In some embodiments, nivolumab is administered at least once a week. In some embodiments, nivolumab is administered once a week. In some embodiments, nivolumab is administered twice a month. In some embodiments, nivolumab is administered three times a month. In some embodiments, nivolumab is administered once a month. In some embodiments, nivolumab is administered in a 28 day cycle, once a week for 3 weeks.

### Diseases

Described herein are methods for treating diseases, where the methods comprise administering a combination of two or more therapies, in particular a combination comprising a tyrosine kinase inhibitor, or a pharmaceutically acceptable salt thereof and an immunotherapeutic agent or a pharmaceutically acceptable salt thereof.

In some embodiments, the disease is a proliferative or a hyper-proliferative condition including, but not limited to cancer, hyperplasias, restenosis, inflammation, immune disorders, cardiac hypertrophy, atherosclerosis, fibrosis, pain, migraine, psoriasis, angiogenesis-related conditions or disorders, proliferation induced after medical conditions, including but not limited to surgery, angioplasty, or other conditions.

Angiogenesis-related conditions or disorders include, but are not limited to, cancers, diabetic retinopathy, proliferative retinopathy, corneal graft rejection, neovascular glaucoma, blindness and macular degeneration, erythema, psoriasis, hemophiliac joints, capillary proliferation within atherosclerotic plaques, keloids, wound granulation, vascular adhesions, rheumatoid arthritis, osteoarthritis, autoimmune diseases, Crohn's disease, restenosis, atherosclerosis, intestinal adhesions, cat scratch disease, ulcers, liver cirrhosis, glomerulonephritis, diabetic nephropathy, malignant nephrosclerosis, thrombotic microangiopathy, organ transplant rejection, glomerulopathy, diabetes, inflammation, and rodegenerative diseases.

In some embodiments, the proliferative disease is cancer. In some embodiments, the proliferative disease is non-cancerous. In some embodiments, the proliferative disease is a benign or malignant tumor. Where hereinbefore and subsequently a tumor, a tumor disease, a carcinoma or a cancer are mentioned, also metastasis in the original organ or tissue and/or in any other location are implied alternatively or in addition, regardless of the location of the tumor and/or metastasis. In some embodiments, the methods include treating, inhibiting and preventing tumor growth.

In some embodiments, the disease is cancer. In some embodiments, the cancer is selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, the cancer is anal cancer, bowel cancer, colon cancer, colorectal cancer, esophageal cancer, gallbladder and biliary tract cancer, gastric cancer, gastrointestinal stromal tumor (gist), gastroesophageal junction cancer, intestinal cancer, liver cancer, neuroendocrine tumors, pancreatic cancer, peritoneal cancer, rectal cancer, small bowel cancer, stomach cancer, or a combination thereof.

In some embodiments, the cancer is gastric cancer. In some embodiments, the cancer is gastroesophageal junction cancer. In some embodiments, the cancer is advanced gastric cancer. In some embodiments, the cancer is advanced gastroesophageal junction cancer. In some embodiments, the cancer is recurrent gastric cancer. In some embodiments, the cancer is recurrent gastroesophageal junction cancer. In some embodiments, the cancer is metastatic gastric cancer. In some embodiments, the cancer is metastatic gastroesophageal junction cancer.

In some embodiments, the cancer comprises one or more lesions. In some embodiments, the lesion is measured before the treatment and either during the treatment or after the treatment or both. In some embodiments, the lesion is measured by radiological assessments using computerized tomography scan or magnetic resonance imaging. In some embodiments, the lesion has reduced in size after the treatment. In some embodiments, the lesion has reduced in size by at least 10%. In some embodiments, the lesion has reduced in size by at least 20%. In some embodiments, the lesion has reduced in size by at least 25%. In some embodiments, the lesion has reduced in size by at least 30%. In some embodiments, the lesion has reduced in size by at least 40%. In some embodiments, the lesion has reduced in size by at least 50%. In some embodiments, the lesion has reduced in size by at least 60%. In some embodiments, the lesion has reduced in size by at least 70%. In some embodiments, the lesion has reduced in size by at least 75%. In some embodiments, the lesion has reduced in size by at least 80%. In some embodiments, the lesion has reduced in size by at least 90%.

Described herein are methods for treating diseases, where the methods comprise administering a combination of two or more therapies, in particular a combination comprising a tyrosine kinase inhibitor, or a pharmaceutically acceptable salt thereof and an immunotherapeutic agent or a pharmaceutically acceptable salt thereof. In some embodiments, the methods are a first line of therapy for treating diseases. In some embodiments, the methods are a second or a third line of therapy after the prior treatment for the disease has failed or substantially failed or the disease is substantially refractory to the first line therapy. In some embodiments, a patient has received at least one line of therapy for treating the disease prior to receiving the combination comprising a tyrosine kinase inhibitor, or a pharmaceutically acceptable salt thereof and an immunotherapeutic agent or a pharmaceutically acceptable salt thereof. In some embodiments, the prior line of therapy may be a line of chemotherapy or immunotherapy.

Described herein are methods for treating diseases, where the methods comprise administering a combination of two or more therapies, in particular a combination comprising a tyrosine kinase inhibitor, or a pharmaceutically acceptable salt thereof and an immunotherapeutic agent or a pharmaceutically acceptable salt thereof. In some embodiments, the methods further comprise administering one or more additional agents selected from the group consisting of anti-cancer agents, anti-proliferative agents, chemotherapeutic agents, immunomodulatory agents, anti-angiogenic agents, anti-inflammatory agents, alkylating agents, steroidal and non-steroidal anti-inflammatory agents, pain relievers, leukotriene antagonists, .beta.2-agonists, anticholinergic agents, hormonal agents, biological agents, immunotherapeutic agents, glucocorticoids, corticosteroid agents, antibacterial agents, antihistamines, anti-malarial agents, anti-viral agents, and antibiotics; and, optionally with radiation therapy.

Described herein are methods for treating diseases, where the methods comprise administering a combination of two or more therapies, in particular a combination comprising a tyrosine kinase inhibitor, or a pharmaceutically acceptable salt thereof and an immunotherapeutic agent or a pharmaceutically acceptable salt thereof. In some embodiments, the combination is administered for at least 2 months. In some embodiments, the combination is administered for about 2 months. In some embodiments, the combination is administered for about 3 months. In some embodiments, the combination is administered for about 4 months. In some embodiments, the combination is administered for about 5 months. In some embodiments, the combination is administered for about 6 months. In some embodiments, the combination is administered for about 7 months. In some embodiments, the combination is administered for about 8 months. In some embodiments, the combination is administered for about 9 months. In some embodiments, the combination is administered for about 10 months. In some embodiments, the combination is administered for about 11 months. In some embodiments, the combination is administered for about 12 months. In some embodiments, the combination is administered for more than 2 months.

It is understood that cancer refer to or describe the physiological condition in mammals that is typically characterized by unregulated cell growth. The cancer may be multi-drug resistant (MDR) or drug-sensitive. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

In various aspects, further examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma. In a still further aspect, the cancer is selected from chronic lymphocytic leukemia, small lymphocytic lymphoma, B-cell non-Hodgkin lymphoma, and large B-cell lymphoma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In one aspect, the cancer can be a cancer selected from cancers of the blood, brain, genitourinary tract, gastrointestinal tract, colon, rectum, breast, kidney, lymphatic system, stomach, lung, pancreas, and skin. In a further aspect, the cancer is selected from prostate cancer, glioblastoma multiforme, endometrial cancer, breast cancer, and colon cancer. In a further aspect, the cancer is selected from a cancer of the breast, ovary, prostate, head, neck, and kidney. In a still further aspect, the cancer is selected from cancers of the blood, brain, genitourinary tract, gastrointestinal tract, colon, rectum, breast, liver, kidney, lymphatic system, stomach, lung, pancreas, and skin. In a yet further aspect, the cancer is selected from a cancer of the lung and liver. In an even further aspect, the cancer is selected from a cancer of the breast, ovary, testes and prostate. In a still further aspect, the cancer is a cancer of the breast. In a yet further aspect, the cancer is a cancer of the ovary. In an even further aspect, the cancer is a cancer of the prostate. In a still further aspect, the cancer is a cancer of the testes.

### Synergy

Described herein are methods for treating diseases, where the methods comprise administering a combination of two or more therapies, in particular a combination comprising a tyrosine kinase inhibitor, or a pharmaceutically acceptable salt thereof and an immunotherapeutic agent or a pharmaceutically acceptable salt thereof.

In some embodiments, the combination of the tyrosine kinase inhibitor, or a pharmaceutically acceptable salt thereof and the immunotherapeutic agent or a pharmaceutically acceptable salt thereof, acts to produce synergistic therapeutic results.

In some embodiments, the combination of the of the tyrosine kinase inhibitor, or a pharmaceutically acceptable salt thereof and the immunotherapeutic agent or a pharmaceutically acceptable salt thereof, results in a joint action where one of the components supplements or enhances the action of the other component to produce an effect greater than that which may be obtained by use of the individual components in equivalent quantities, or produce effects that could not be obtained with safe quantities of the other components individually.

In some embodiments, the tyrosine kinase inhibitor, or a pharmaceutically acceptable salt thereof and the immunotherapeutic agent or a pharmaceutically acceptable salt thereof, work together to produce a therapeutic effect greater than the sum of their individual effects.

In some embodiments, the interaction of the tyrosine kinase inhibitor, or a pharmaceutically acceptable salt thereof and the immunotherapeutic agent or a pharmaceutically acceptable salt thereof is such that the addition of one compound, results in less of the other compound being required, to achieve the same therapeutic effect.

In some embodiments, administration of the tyrosine kinase inhibitor or a pharmaceutically acceptable salt thereof, results in the need for a smaller dose of the immunotherapeutic agent or a pharmaceutically acceptable salt thereof.

In some embodiments, administration of the immunotherapeutic agent or a pharmaceutically acceptable salt thereof, results in the need for a smaller dose of the tyrosine kinase inhibitor or a pharmaceutically acceptable salt thereof.

### Pharmaceutical Compositions

In one aspect, the disclosure relates to pharmaceutical compositions comprising the compounds of the disclosure. That is, a pharmaceutical composition can be provided comprising a therapeutically effective amount of apatinib and a therapeutically effective amount of an immunotherapeutic agent and a pharmaceutically acceptable carrier.

In certain aspects, the disclosed pharmaceutical compositions comprise the disclosed compounds (such as apatinib and am immunotherapeutic agent) (including pharmaceutically acceptable salt(s) thereof) as active ingredients, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. A particularly preferred salt is mesylate. Salts derived from such inorganic bases include aluminum, ammonium, calcium, copper (-ic and -ous), ferric, ferrous, lithium, magnesium, manganese (-ic and -ous), potassium, sodium, zinc and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

In practice, the compounds of the disclosure, or pharmaceutically acceptable salts thereof, of this disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this disclosure can include a pharmaceutically acceptable carrier, apatinib and an immunotherapeutic agent, or a pharmaceutically acceptable salt of apatinib and/or the immunotherapeutic agent. The compounds of the disclosure, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this disclosure can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present disclosure comprise a compound of the disclosure (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present disclosure suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present disclosure can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in moulds.

In addition, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the disclosure, and/or pharmaceutically acceptable salts thereof, can also be prepared in powder or liquid concentrate form.

It is understood that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

In one embodiment of the present disclosure, a composition of a combination therapy of the disclosure may be administered in a single dosage form comprising all the therapeutically active agents together.

In another embodiment, the combination therapy of the present disclosure comprises more than two compositions contained in separate containers, and these at least two compositions may be administered separately, either simultaneously or sequentially.

### Kits

In some embodiments, kits are provided, comprising:
a) a tyrosine kinase inhibitor, or a pharmaceutically acceptable salt thereof; and
b) an immunotherapeutic agent, or a pharmaceutically acceptable salt thereof;
   wherein the kits are for treating diseases.

In some embodiments, kits are provided, comprising:
a) rivoceranib, or a pharmaceutically acceptable salt thereof; and
b) an immunotherapeutic agent, or a pharmaceutically acceptable salt thereof;
   wherein the kits are for treating diseases.

In some embodiments, kits are provided, comprising:
a) a tyrosine kinase inhibitor, or a pharmaceutically acceptable salt thereof; and
b) pembrolizumab;
   wherein the kits are for treating diseases.

In some embodiments, kits are provided, comprising:
a) a tyrosine kinase inhibitor, or a pharmaceutically acceptable salt thereof; and
b) nivolumab;
   wherein the kits are for treating diseases.

In some embodiments, kits are provided, comprising:
a) a tyrosine kinase inhibitor, or a pharmaceutically acceptable salt thereof; and
b) an immunotherapeutic agent, or a pharmaceutically acceptable salt thereof;
   wherein the kits are for treating cancer.

In some embodiments, kits are provided, comprising:
a) rivoceranib, or a pharmaceutically acceptable salt thereof; and
b) an immunotherapeutic agent, or a pharmaceutically acceptable salt thereof;
   wherein the kits are for treating cancer.

In some embodiments, kits are provided, comprising:
a) a tyrosine kinase inhibitor, or a pharmaceutically acceptable salt thereof; and
b) pembrolizumab;
   wherein the kits are for treating cancer.

In some embodiments, kits are provided, comprising:
a) a tyrosine kinase inhibitor, or a pharmaceutically acceptable salt thereof; and
b) nivolumab;
   wherein the kits are for treating cancer.

In some embodiments, kits are provided, comprising:
a) a tyrosine kinase inhibitor, or a pharmaceutically acceptable salt thereof; for once daily oral administration; and
b) an immunotherapeutic agent, or a pharmaceutically acceptable salt thereof, for once weekly iv administration;
   wherein the kits are for treating diseases.

In some embodiments, kits are provided, comprising a tyrosine kinase inhibitor, or a pharmaceutically acceptable salt thereof, present in an amount effective to enhance the efficacy of the immunotherapeutic agent to treat diseases.

In some embodiments, kits are provided, comprising an immunotherapeutic agent, or a pharmaceutically acceptable salt thereof, present in an amount effective to enhance the efficacy of the tyrosine kinase inhibitor to treat diseases.

In some embodiments, kits are provided, comprising:
a) a tyrosine kinase inhibitor, or a pharmaceutically acceptable salt thereof;
b) an immunotherapeutic agent, or a pharmaceutically acceptable salt thereof;
c) a corticosteroid;
d) an antihistamine; and
e) an H₂ receptor antagonist;
   wherein the kits are for treating diseases

In some embodiments, packaged pharmaceutical therapies are provided, comprising:
a) a tyrosine kinase inhibitor, or a pharmaceutically acceptable salt thereof;
b) an immunotherapeutic agent, or a pharmaceutically acceptable salt thereof;
c) a container; and
d) instructions for use of the therapy to treat a disease or condition in a mammal.

A kit for treating cancer, comprising
i) rivoceranib, or a pharmaceutically acceptable salt thereof, for once daily oral administration; and
ii) pembrolizumab for iv administration.

A kit for treating cancer, comprising
i) rivoceranib, or a pharmaceutically acceptable salt thereof, for once daily oral administration; and
ii) nivolumab for iv administration.

### EXAMPLES

### Example 1

Synergistic Tumor Growth Inhibition of Combinatorial Treatment of Apatinib Mesylate and Anti-muPD-1 Antibody in Lung Carcinoma Syngeneic Mouse Model

### Experimental Animals

40 female C57BL/6 mice purchased from Jackson Laboratories were enrolled on the study. Animals were housed for stabilization period. Animals were housed in individual HEPA ventilated cages (Innocage® IVC, Innovive USA). Fluorescent lighting was provided on a 12-hour cycle. Temperature and humidity was monitored and recorded daily and maintained to the maximum extent possible between 68-74° F. (20-23° C.) and 30-70% humidity, respectively. 2920×.10 18% soy irradiated rodent feed (Harlan) and autoclaved acidified water (pH 2.5-3) was provided ad libitum.

### Experimental Procedures

For inoculation, LL/2 cell viability was at 98%. Cryo vials containing LL/2 cells were thawed and prepared for injection into mice. On study day −3 cells were washed in PBS, counted, and re-suspended in cold PBS at concentrations of 250,000 viable cells/100 μL. Cell suspensions were mixed with PBS and kept on ice during transport to the vivarium. Cells for injections were prepared by withdrawing the PBS-cell mixture into a chilled 1 mL Lure-lok syringe fitted with a 26 7/8 G (0.5 mm×22 mm) needle. Animals were shaved prior to injection. One mouse at a time was immobilized and the site of injection was disinfected with an alcohol swab. 100 μL of the cell suspension was injected subcutaneously into the rear flank.

Forty mice were enrolled in the study. Ten mice were randomly allocated to each of 4 different study groups: vehicle control, anti-muPD-1 antibody, apatinib mesylate, and apatinib+anti-muPD-1 combination. Randomization was performed in the Study Log software on day 1 when the mean tumor volume was between 73-80 mm³. 300 mg/kg of apatinib mesylate formulated by mixing with 0.5% carboxymethylcellulose solution was dosed daily (q.d.) and orally (p.o.), while 10 mg/kg of anti-muPD-1 antibody (RMP1-14) solution was dosed 2 times a week (b.i.w.) and intraperitoneally (i.p).

Animals were monitored weekly for palpable tumors, or any changes in appearance or behavior. Once tumors were palpable, they were measured 3 times a week using calipers. Tumor volume was calculated using the following equation (longest diameter×shortest diameter 2)/2.

Body weight was measured at least three times a week. All measurements were performed prior to dosing of test articles on day of measurement during the treatment period. If body weight loss of >15% was observed, animal was given a dosing holiday until weight loss was <10%. If body weight loss of >20% was observed, animal was sacrificed for humane reasons as per IACUC protocol regulations.

At the end of the study (day 25), spleens and tumors were isolated. Any animal that had to be euthanized prior to the end of the study or due to tumor volume reaching 3000 mm³ were dosed, sampled.

### Results

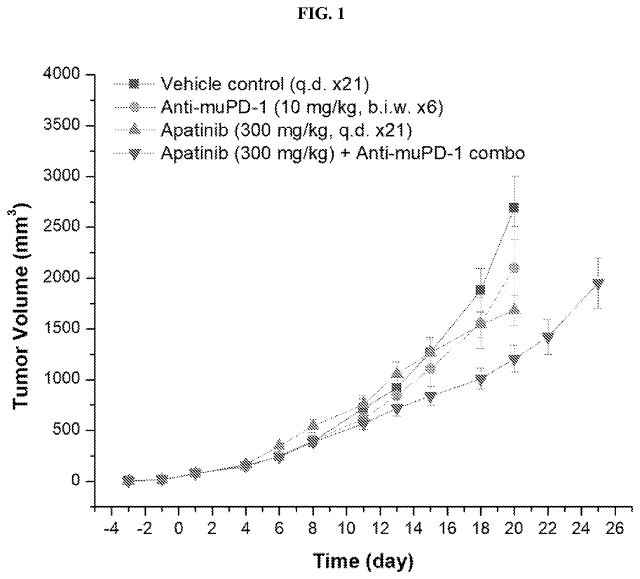

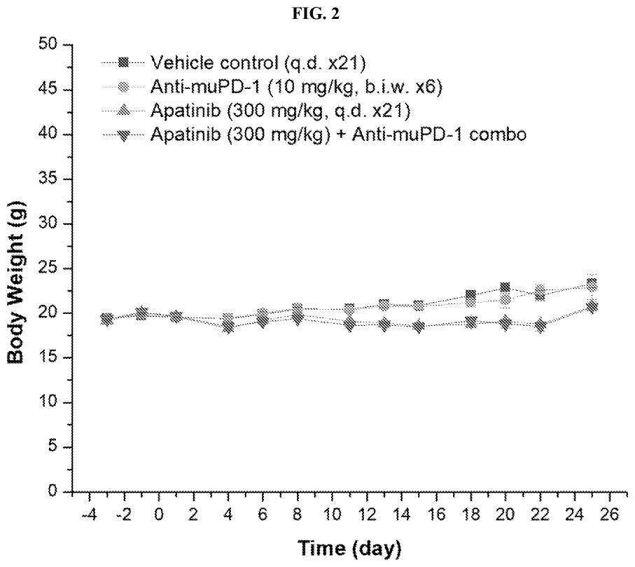

As shown in FIG. 1 and TABLE 3, a statistically extremely significant difference (p<0.0001) in tumor growth inhibition was found in animals with daily oral treatment of apatinib mesylate (300 mg/kg) in combination with twice a week IP treatment of anti-muPD-1 antibody vs. vehicle treated mice after 18 days. On the other hand, there was no significant difference in tumor growth inhibition between anti-muPD-1 antibody vs. vehicle treated group on day 18, 22, 25. These conclusions are based on two-way ANOVA (main column effect) analysis with Dunnet's comparison test against the vehicle group. As shown in FIG. 2, there was no significant body weight loss from all of the experimental groups in entire dosing period.

TABLE 3
Tumor growth inhibition percentage in day 20 demonstrating
synergistic anti-tumor efficacy of apatinib mesylate
combining with anti-muPD-1 antibody on mouse LL/2 lung
carcinoma transplanted in syngeneic mice.

| Experimental groups | % Tumor growth inhibition¹ | p value² |
|---|---|---|
| Vehicle control | — | — |
| Anti-muPD-1 (10 mg/kg) | 22% | <0.05 |
| Apatinib (300 mg/kg) | 37% | <0.0001 |
| Apatinib + Anti-muPD-1 combo | 55% | <0.0001 |

¹% Tumor growth inhibition = (([mean day 20 control] − [mean day 0 control]) − ([mean day 20 group] − [mean day 0 group]))/([mean day 20 control] − [mean day 0 control]) * 100%
²Two-way ANOVA (main column effect) analysis with Dunnet's comparison test against the vehicle group

### Example 2

### Ongoing Phase I/II Clinical Trials of Rivoceranib/Nivolumab Combination Therapy

Overall Design:

A Phase I study to evaluate the safety, tolerability, and efficacy of adding rivoceranib to ongoing nivolumab treatment in patients with unresectable or metastatic cancer.

Objectives:

Primary objectives are to evaluate safety & tolerability, efficacy by objective response rate, best overall response, time to response, duration of response, disease control rate, and duration of disease control. Secondary objectives are to evaluate efficacy as measured by overall survival, progression-free survival and event-free survival.

Patients:

Approximately 9-18 patients in Phase I. Up to 12 additional patients in Part II. Eligible patients have advanced unresectable or metastatic disease, with documented primary diagnosis of solid tumor cancer inclusive of gastric adenocarcinoma, renal cell carcinoma, melanoma, non-small cell lung cancer, and breast or other solid tumor for which anti-VEGFR-2 targeted therapy could be applicable. Patients will have received at least three prior doses of nivolumab treatment and are continuing nivolumab therapy.

Does Determination:

Rivoceranib 685 mg was studied as a single agent in patients with advanced gastric cancer in Phase 2 and Phase 3 studies and demonstrated efficacy against gastric cancer with manageable toxicity, establishing once daily rivoceranib 685 mg as the recommended dose for gastric cancer. A Phase 3 study is ongoing, using a starting dose of 700 mg rivoceranib. 400 mg was selected as the starting dose. Nivolumab (240 mg, administered once every 2 weeks) forms the primary treatment, supplemented with varying amounts of rivoceranib. The combined use of rivoceranib and nivolumab enhances the antitumor activity of both agents for improved progression-free and overall survival.

Study Duration:

The total duration of the study will be approximately 12 months: 6 months of recruitment plus 6 months of treatment.

Dose Escalation (Phase I):

A sequential evaluation of 3 subjects at each dose level is performed with escalating doses of rivoceranib, starting at 400 mg, escalating up to 700 mg, in combination with nivolumab 240 mg administered IV every 2 weeks, to determine the maximum tolerated dose, as described in Table 4.

Initially, three subjects receive daily doses of rivoceranib at the starting dose (400 mg) for a 28-day observation period for dose-limiting toxicity (DLT) evaluation. If no DLT occurs, three additional subjects are enrolled to the next dosing level. If one DLT event occurs, an additional three subjects are enrolled to the same cohort. If no additional DLT occurs, the next dosing cohort begins; if a second DLT occurs, that dose is considered intolerable.

TABLE 4

| | nivolumab | Rivoceranib |
|---|---|---|
| Cohort O2 | 240 mg Q2 weeks IV | 200 mg QD PO |
| Cohort O1 | | 300 mg QD PO |
| Cohort A | | 400 mg QD PO |
| Cohort B | | 500 mg QD PO |
| Cohort C | | 600 mg QD PO |
| Cohort D | | 700 mg QD PO |

Expansion (Phase II):

Up to 20 patients (with unresectable and metastatic cancer, including subjects with Angiosarcoma, Leiomyosarcoma, and Synovial Sarcoma, and Alveolar Soft Part Sarcoma) begin treatment at the maximum tolerated dose determined in Phase I, in combination with nivolumab 240 mg, administered IV once every 2 weeks.

Study Treatment Details:

Rivoceranib is administered as the mesylate salt of its free base, provided as 100 mg and 200 mg tablets in PVC heat-sealed, foil-laminated blister packs. Prior studies refer to doses of Rivoceranib as the weight of the mesylate salt. Rivoceranib doses as provided in this example, are given as the amount of freebase rather than the mesylate salt. The freebase dosage is approximately 81% of the mesylate dosage. The formulation is the same. Referring to Rivoceranib dose strength as freebase aligns with standards for referencing total active product.

### Preliminary Results

A classic 3+3 dose escalation has been completed (Part 1) and the study is currently ongoing with an extension period (Part 2). Herein, we present the safety and preliminary efficacy.

Methods:

To date, 10 subjects were enrolled in Part 1 while 3 subjects have been enrolled in Part 2. Median number of prior lines of therapy were 3 and 1, respectively, for the two study segments. The major inclusion criteria specified that patients must have received at least 3 doses of nivolumab and are continuing nivolumab therapy. Escalating orally adminstered doses of rivoceranib at 400, 600, and 700 mg once a day was proposed in combination with 240 mg of nivolumab administered intravenously once every 2 weeks to determine the maximum tolerated dose.

Results:

300 mg rivoceranib was determined as RP2D for Part 2. 5/13 patients had G2/3 hypertension. Common treatment-related AEs were hypertension, hand and foot syndrome, and nausea and immune-related AEs were hypothyroidism and diarrhea. Notably, in 10 evaluable patients, 3 patients had PD and 4 patients showed tumor shrinkage (5-29%). A patient with Malignant Spindled/Epitheloid Sarcoma, who had PD during previous nivolumab treatment has shown tumor reduction, so far, of 9%. A patient with Gastric Cancer, who had stable disease (a 4% increase) after 2 months of nivolumab showed a reduction of 29%, 2 months after introduction of rivoceranib. 2 patients have had stable disease for more than 8 months. Conclusions: Preliminary results indicate the potential clinical benefit of a 300 mg starting dose in combination with 240 mg of nivolumab in unresectable/metastatic solid tumors with a tolerable safety profile. Efficacy of ongoing and future patients in Part 2 will be further evaluated.

### Example 3

### Ongoing Phase I/II Clinical Trials of Rivoceranib/Pembrolizumab Combination Therapy

Overall Design:

A Phase I/II, non-randomized, open label study of the safety and efficacy of rivoceranib administered to patients with advanced malignancies to improve sensitivity to pembrolizumab in the second- or later-line setting.

Objectives:

The general objective is to evaluate the clinical activity of rivoceranib in combination with pembrolizumab in subjects with select advanced malignancies. Phase I objectives are to determine the recommended phase II dose and establish the toxicity profile of the pembrolizumab-rivoceranib combination. Phase II objectives are to determine the efficacy of the combination, in terms of
objective response rate;
objective response rate by clinical benefit rate;
progression free survival;
overall survival;
adverse events and serious adverse events.

Patients:

Up to 119 patients are enrolled in the study, with advanced malignancies in urothelial carcinoma, MSI-H or dMMR Solid Tumors or gastric or gastroesophageal junction adenocarcinoma. Phase 1: for a 3+3 dose escalation design with up to 5 escalation dose levels (and 1 de-escalation level) involving 4-30 patients. Phase 2: up to 89 patients will be recruited in three cohorts, 25 in the urothelial cohort, 38 in the MSI-H cohort, and 26 in the gastric & GEJ cohort. Eligible patients will have one of the following advanced solid malignancies, which qualifies for standard of care pembrolizumab treatment:
Locally advanced or metastatic urothelial carcinoma, only in the second- or later-line setting who have received first-line platinum-based chemotherapy;
Unresectable or metastatic microsatellite instability—high or mismatch repair deficient solid tumors that have progressed during or following prior treatment and have no satisfactory alternative treatment options, including colorectal cancer that has progressed following treatment with a fluoropyrimidine, oxaliplatin, and irinotecan;
Recurrent locally advanced or metastatic, gastric or gastroesophageal junction adenocarcinoma that have progressed on or after two or more systemic therapies, including fluoropyrimidine- and platinum-containing chemotherapy and, if appropriate, HER2/neu-targeted therapy.

Dose Determination:

Rivoceranib 685 mg was studied as a single agent in patients with advanced gastric cancer in Phase 2 and Phase 3 studies and demonstrated efficacy against gastric cancer with manageable toxicity, establishing once daily rivoceranib 685 mg as the recommended dose for gastric cancer. A Phase 3 study is ongoing, using a starting dose of 700 mg rivoceranib. However, given this is the first-in-human evaluation of this combination and patients with bladder cancer have yet to be exposed to rivoceranib, the starting daily dose of 300 mg rivoceranib was deemed appropriate to ensure patient safety while maintaining efficacy. Thus, pembrolizumab (200 mg, administered once every 3 weeks) forms the primary treatment, supplemented with varying amounts of rivoceranib. The combined use of rivoceranib and pembrolizumab enhances the antitumor activity of both agents for improved progression-free and overall survival.

Study Duration:

The total duration of the study will be up to 6 years. The estimated duration of accrual is 6 months for Phase 1 and 28 months for Phase 2. The study plans for 1 year for enrollment in Phase 1 and first part of Phase 2, 2 years of enrollment for second part of Phase 2 and up to 3 more years of active treatment.

Dose escalation (Phase I):

Phase I assesses the safety of combining up to 5 increasing dose levels of rivoceranib, administered orally, with a fixed dose of pembrolizumab (200 mg) administered intravenously every three weeks (see Table 5) and will determine the recommended phase II dose. The starting dose is 300 mg oral rivoceranib daily, escalating to 400 mg, 500 mg, 600 mg and 700 mg, or deescalating to 200 mg. A standard 3+3 dose escalation design is used, with a minimum of 3 (and up to 6) evaluable subjects recruited to each dose level (starting with dose level 1).

TABLE 5

| | Pembrolizumab | Rivoceranib |
|---|---|---|
| Dose Level −1 | 200 mg Q3 weeks IV | 200 mg QD PO |
| Dose Level 1 (starting dose) | | 300 mg QD PO |
| Dose Level 2 | | 400 mg QD PO |
| Dose Level 3 | | 500 mg QD PO |
| Dose Level 4 | | 600 mg QD PO |
| Dose Level 5 | | 700 mg QD PO |

The study follows a 3+3 dose-escalation scheme until maximum tolerated dose is established; dose escalation, dose de-escalation, or dose level expansion proceeds based on the occurrence of DLTs at each dose level

Evaluation of a cohort of at least 3 patients completing Cycle 1 at that dose level is required prior to determining the next dose level for the next cohort. If a DLT is observed in 1 of 3 patients, 3 additional patients are enrolled at the same dose. If a DLT is observed in 1 of the 6 patients,—no additional patients are required and—the next dose level is opened. If a DLT is observed in ≥2 patients,—the previous dose level is the MTD.

The recommended phase II dose is determined from the 3+3 algorithm or when six patients have been treated at the maximum dose with one or no dose limiting toxicities observed. Phase II assesses the efficacy of the recommended phase II dose of rivoceranib in combination with pembrolizumab and provides additional safety and tolerability data in the three disease-specific cohorts.

Study Treatment Details:

Rivoceranib is administered as the mesylate salt of its free base. This example refers to the rivoceranib dose strength as the amount of rivoceranib freebase rather than of amount of the rivoceranib mesylate salt. The freebase dosage is approximately 81% of the mesylate dosage with identical formulation. Referring to rivoceranib's dose strength as the freebase aligns with standards for referencing total active product. Rivoceranib is provided as film-coated oral tablets in 200 mg and 100 mg strength (248 mg and 124 mg of rivoceranib mesylate). Components used in the manufacture of the product are listed in Table 6 below. The tablets are packaged in PVDC, heat-sealed foil-laminated blister packs.

TABLE 6

| | | Amount/ | Amount/ |
|---|---|---|---|
| | | table | table |
| | | (100 mg | (200 mg |
| Componant | Function | freebase) | freebase) |
| Rivoceranib mesylate | Drug Substance | 124.2 | 248.4 |
| Pregelatinized starch | Diluent | 49.7 | 99.3 |
| Microcrystalline cellulose | Diluent | 39.7 | 79.5 |
| Sodium starch glycolate | Disintegrant | 14.9 | 29.8 |
| Povidone (K-30) | Binder | 3.1 | 6.3 |
| Colloidal silicon dioxide | Glidant | 4.0 | 8.0 |
| Magnesium stearate | Lubricant | 4.0 | 8.0 |
| Opadry white | Coating Material | 6.8 | 13.5 |
| Alcohol* | Granulation Fluid | — | — |
| Purified water* | Coating Fluid | — | — |
| | TOTAL | 246.3 | 492.7 |

*Removed during manufacturing process.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating cancer, comprising administering:
a) rivoceranib, or a pharmaceutically acceptable salt thereof; and
b) pembrolizumab.

2. A method for treating cancer, comprising administering:
a) rivoceranib, or a pharmaceutically acceptable salt thereof; and
b) nivolumab.

3. The method of claim 1 or 2, comprising administering a pharmaceutically acceptable salt of rivoceranib.

4. The method of claim 3, wherein the pharmaceutically acceptable salt of rivoceranib is a mesylate salt.

5. The method of any one of claims 1 to 3, wherein the rivoceranib is administered in an amount of from 100 mg to 1000 mg.

6. The method of any one of claims 1 to 3, wherein the rivoceranib is administered in an amount of from 150 mg to 800 mg.

7. The method of any one of claims 1 to 3, wherein the rivoceranib is administered in an amount of from 200 mg to 700 mg.

8. The method of any one of claims 1 to 3, wherein the rivoceranib is administered in an amount of less than 700 mg.

9. The method of any one of claims 1 to 3, wherein the rivoceranib is administered in an amount of about 200 mg.

10. The method any one of claims 1 to 3, wherein the rivoceranib is administered in an amount of about 300 mg.

11. The method of any one of claims 1 to 3, wherein the rivoceranib is administered in an amount of about 400 mg.

12. The method any one of claims 1 to 3, wherein the rivoceranib is administered in an amount of about 500 mg.

13. The method any one of claims 1 to 3, wherein the rivoceranib is administered in an amount of about 600 mg.

14. The method of any one of claims 1 to 3, wherein the rivoceranib is administered in an amount of about 685 mg.

15. The method of any one of claims 1 to 3, wherein the total daily dose of the rivoceranib is less than 700 mg.

16. The method of any one of claims 1 to 3, wherein the total daily dose of the rivoceranib is less than 685 mg.

17. The method of any one of claims 1 to 16, wherein the rivoceranib is administered orally.

18. The method of any one of claims 1 to 16, wherein the rivoceranib is administered as a dried powder, a liquid, a capsule, a pellet or a tablet.

19. The method of any one of claims 1 to 16, wherein the rivoceranib is administered as a tablet.

20. The method of claim 19, wherein the tablet is a film coated tablet.

21. The method of claim 19, wherein the tablet comprises the rivoceranib in an amount of about 100 mg.

22. The method of claim 19, wherein the tablet comprises the rivoceranib in an amount of about 200 mg.

23. The method of claim 19, wherein the tablet further comprises one or more of pregelatinized starch, microcrystalline cellulose, sodium starch glycolate, povidone (K-30), colloidal silicon dioxide, magnesium stearate and Opadry white.

24. The method of any one of claims 1 to 23, wherein the rivoceranib is administered once daily.

25. The method any one of claims 1 to 23, wherein the rivoceranib is administered twice daily.

26. The method any one of claims 1 to 23, comprising administering pembrolizumab.

27. The method any one of claims 1 to 23, comprising administering pembrolizumab in a dosage amount of about 200 mg.

28. The method of claim 1, wherein the pembrolizumab is administered in a dosage amount of 150 mg to 250 mg.

29. The method of claim 1, wherein the pembrolizumab is administered orally or parenterally.

30. The method of claim 1, wherein the pembrolizumab is administered parenterally.

31. The method of claim 30, wherein the parenteral administration is selected from intravenous, intradermal, intramuscular or subcutaneous administration.

32. The method of claim 1, wherein the pembrolizumab is administered intravenously.

33. The method of claim 1, wherein the pembrolizumab is administered after the administration of the rivoceranib.

34. The method of claim 1, wherein the pembrolizumab is administered about an hour after the administration of the rivoceranib.

35. The method of claim 1, wherein the pembrolizumab is administered over a period of less than one hour.

36. The method of claim 1, wherein the pembrolizumab is administered over a period of about an hour.

37. The method of claim 1, wherein the pembrolizumab is administered over a period of 30-60 minutes.

38. The method of claim 1, wherein the pembrolizumab is administered no more than once a week.

39. The method of claim 1, wherein the pembrolizumab is administered at least once a week.

40. The method of claim 1, wherein the pembrolizumab is administered once a week.

41. The method of claim 1, wherein the pembrolizumab is administered every three weeks.

42. The method of claim 1, wherein the pembrolizumab is administered once a month.

43. The method of claim 1, wherein the pembrolizumab is administered twice a month.

44. The method of claim 1, wherein the pembrolizumab is administered three times a month.

45. The method of claim 2, comprising administering nivolumab.

46. The method of claim 2, comprising administering nivolumab in a dosage amount of about 240 mg.

47. The method of claim 2, wherein the nivolumab is administered in a dosage amount of 200 mg to 300 mg.

48. The method of claim 2, wherein the nivolumab is administered orally or parenterally.

49. The method of claim 2, wherein the nivolumab is administered parenterally.

50. The method of claim 49, wherein the parenteral administration is selected from intravenous, intradermal, intramuscular or subcutaneous administration.

51. The method of claim 2, wherein the nivolumab is administered intravenously.

52. The method of claim 2, wherein the nivolumab is administered after the administration of the rivoceranib.

53. The method of claim 2, wherein the nivolumab is administered about an hour after the administration of the rivoceranib.

54. The method of claim 2, wherein the nivolumab is administered over a period of less than one hour.

55. The method of claim 2, wherein the nivolumab is administered over a period of about an hour.

56. The method of claim 2, wherein the nivolumab is administered over a period of 30-60 minutes.

57. The method of claim 2, wherein the nivolumab is administered no more than once a week.

58. The method of claim 2, wherein the nivolumab is administered at least once a week.

59. The method of claim 2, wherein the nivolumab is administered every two weeks.

60. The method of claim 2, wherein the nivolumab is administered once a week.

61. The method of claim 2, wherein the nivolumab is administered once a month.

62. The method of claim 2, wherein the nivolumab is administered twice a month.

63. The method of claim 2, wherein the nivolumab is administered three times a month.

64. The method of any one of claims 1 to 63, wherein the cancer is selected from lung cancer, small-cell lung cancer, non-small cell lung cancer, carcinoma, lymphoma, blastoma, sarcoma, leukemia, breast cancer, prostate cancer, colon cancer, squamous cell cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

65. The method of claim 1, wherein the cancer is melanoma or non-small cell lung cancer.

66. The method of claim 2, wherein the cancer is non-small cell lung cancer, advanced small cell lung cancer, metastatic melanoma, kidney cancer (renal cell carcinoma), advanced renal cell carcinoma, squamous cell carcinoma, liver cancer, (hepatocellular carcinoma), bladder cancer (urothelial carcinoma), colon cancer, or Hodgkin lymphoma.

67. The method of any one of claims 1 to 65, further comprising administering radiation therapy.

68. The method any one of claims 1 to 67, wherein the cancer comprises a lesion.

69. The method of claim 68, wherein the lesion is measured before the treatment and either during the treatment or after the treatment or both.

70. The method of claim 69, wherein the lesion is measured by radiological assessments using computerized tomography scan or magnetic resonance imaging.

71. The method of claim 68, wherein the lesion has reduced in size after the treatment.

72. The method of claim 68, wherein the lesion has reduced in size by at least 10%.

73. The method of claim 68, wherein the lesion has reduced in size by at least 20%.

74. The method of claim 68, wherein the lesion has reduced in size by at least 50%.

75. The method of claim 68, wherein the lesion has reduced in size by at least 75%.

76. A method for treating cancer, comprising administering a combination of
a) a tyrosine kinase inhibitor, or a pharmaceutically acceptable salt thereof; and
b) an immunotherapeutic agent, or a pharmaceutically acceptable salt thereof.

77. The method of claim 76, wherein the tyrosine kinase inhibitor is a vascular endothelial growth factor receptor (VEGF) inhibitor.

78. The method of claim 77, wherein the tyrosine kinase inhibitor is a selective vascular endothelial growth factor receptor-2 (VEGF2) inhibitor.

79. The method of claim 76, wherein the tyrosine kinase inhibitor is afatinib, alectinib, apatinib, axitinib, bosutinib, brigatinib, canertinib, crizotinib, ceritinib, dasatinib, danusertib, dabrafenib, erlotinib, gefitinib, ibrutinib, imatinib, lapatinib, lenvatinib, neratinib, nilotinib, nintedanib, osimertinib, palbociclib, pazopanib, pegaptanib, ponatinib, rebastinib, regorafenib, ribociclib, rivoceranib, ruxolitinib, semaxinib, sorafenib, sunitinib, tivozanib, trametinib, tofacitinib, vandetanib, vatalanib, vemurafenib or vismodegib.

80. The method of claim 79, wherein the tyrosine kinase inhibitor is rivoceranib.

81. The method of claim 80, wherein the tyrosine kinase inhibitor is rivoceranib mesylate.

82. The method of any one of claims 76 to 81, wherein the tyrosine kinase inhibitor is administered in an amount of from 150 mg to 800 mg.

83. The method of any one of claims 76 to 81, wherein the tyrosine kinase inhibitor is administered in an amount of from 200 mg to 700 mg.

84. The method of any one of claims 76 to 81, wherein the tyrosine kinase inhibitor is administered in an amount of less than 700 mg.

85. The method of any one of claims 76 to 81, wherein the tyrosine kinase inhibitor is administered in an amount of about 200 mg.

86. The method of any one of claims 76 to 81, wherein the tyrosine kinase inhibitor is administered in an amount of about 300 mg.

87. The method of any one of claims 76 to 81, wherein the tyrosine kinase inhibitor is administered in an amount of about 400 mg.

88. The method of any one of claims 76 to 81, wherein the tyrosine kinase inhibitor is administered in an amount of about 500 mg.

89. The method of any one of claims 76 to 81, wherein the tyrosine kinase inhibitor is administered in an amount of about 600 mg.

90. The method of any one of claims 76 to 81, wherein the total daily dose of the tyrosine kinase inhibitor is less than 700 mg.

91. The method of any one of claims 76 to 81, wherein the total daily dose of the tyrosine kinase inhibitor is less than 685 mg.

92. The method of any one of claims 76 to 91, wherein the tyrosine kinase inhibitor is administered orally.

93. The method of any one of claims 76 to 92, wherein the tyrosine kinase inhibitor is administered as a tablet.

94. The method of any one of claims 76 to 93, wherein the tyrosine kinase inhibitor is administered once daily.

95. The method of any one of claims 76 to 93, wherein the tyrosine kinase inhibitor is administered twice daily.

96. The method of any one of claims 76 to 93, wherein the immunotherapeutic agent is a PD-1 inhibitor.

97. The method of claim 96, wherein the PD-1 inhibitor is selected from nivolumab (Opdivo®), pembrolizumab (Keytruda®), MEDI0680 (AMP-514), AMP-224, AMP-514 (Amplimmune), BGB-A317, PDR001, REGN2810, JS001, AGEN2034, and variants and biosimilars thereof.

98. The method of claim 97, wherein the PD-1 inhibitor is selected from nivolumab (Opdivo®), pembrolizumab (Keytruda®), and variants and biosimilars thereof.

99. The method of any one of claims 76 to 98, wherein the immunotherapeutic agent is administered in a dosage amount of 200 mg to 300 mg.

100. The method of any one of claims 76 to 99, wherein the immunotherapeutic agent is administered orally or parenterally.

101. The method of any one of claims 76 to 99, wherein the immunotherapeutic agent is administered parenterally.

102. The method of any one of claims 76 to 99, wherein the parenteral administration is selected from intravenous, intradermal, intramuscular or subcutaneous administration.

103. The method of any one of claims 76 to 99, wherein the immunotherapeutic agent is administered intravenously.

104. The method of any one of claims 76 to 103, wherein the immunotherapeutic agent is administered after the administration of the tyrosine kinase inhibitor.

105. The method of any one of claims 76 to 103, wherein the immunotherapeutic agent is administered about an hour after the administration of the tyrosine kinase inhibitor.

106. The method of any one of claims 76 to 103, wherein the immunotherapeutic agent is administered over a period of less than one hour.

107. The method of any one of claims 76 to 103, wherein the immunotherapeutic agent is administered over a period of about an hour.

108. The method of any one of claims 76 to 103, wherein the immunotherapeutic agent is administered over a period of 30-60 minutes.

109. The method of any one of claims 76 to 103, wherein the immunotherapeutic agent is administered no more than once a week.

110. The method of any one of claims 76 to 103, wherein the immunotherapeutic agent is administered at least once a week.

111. The method of any one of claims 76 to 103, wherein the immunotherapeutic agent is administered every two weeks.

112. The method of any one of claims 76 to 103, wherein the immunotherapeutic agent is administered once a week.

113. The method of any one of claims 76 to 103, wherein the immunotherapeutic agent is administered once a month.

114. The method any one of claims 76 to 103, wherein the immunotherapeutic agent is administered twice a month.

115. The method any one of claims 76 to 103, wherein the immunotherapeutic agent is administered three times a month.

116. A method for treating cancer, comprising administering a combination of
a) rivoceranib, or a pharmaceutically acceptable salt thereof; and
b) no more than 80 mg/m² pembrolizumab.

117. A method for treating cancer, comprising administering a combination of
a) no more than 685 mg rivoceranib, or a pharmaceutically acceptable salt thereof; and
b) pembrolizumab.

118. A method for treating cancer, comprising administering a combination of
a) no more than 685 mg rivoceranib, or a pharmaceutically acceptable salt thereof; and
b) no more than 80 mg/m² pembrolizumab.

119. A method for treating cancer, comprising administering a combination of
a) rivoceranib, or a pharmaceutically acceptable salt thereof; and
b) pembrolizumab;
wherein the rivoceranib and the pembrolizumab act synergistically.

120. A method for treating cancer, comprising administering a combination of
a) rivoceranib, or a pharmaceutically acceptable salt thereof; and
b) no more than 80 mg/m² nivolumab.

121. A method for treating cancer, comprising administering a combination of
a) no more than 685 mg rivoceranib, or a pharmaceutically acceptable salt thereof; and
b) nivolumab.

122. A method for treating cancer, comprising administering a combination of
a) no more than 685 mg rivoceranib, or a pharmaceutically acceptable salt thereof; and
b) no more than 80 mg/m² nivolumab.

123. A method for treating cancer, comprising administering a combination of
a) rivoceranib, or a pharmaceutically acceptable salt thereof; and
b) nivolumab;
wherein the rivoceranib and the nivolumab act synergistically.
